(19) United States
(12) Patent Application Publication
(10) Pub. No.: US 2022/0118261 A1
(43) Pub. Date: Apr. 21, 2022

(54) CURLED SHAFT TEMPORARY PACING LEAD

(71) Applicants: Wesley Robert Pedersen, Minneapolis, MN (US); Paul Sorajja, Minneapolis, MN (US); Brett Allyn Williams, North Oaks, MN (US); William J. Drasler, Minnetonka, MN (US)

(72) Inventors: Wesley Robert Pedersen, Minneapolis, MN (US); Paul Sorajja, Minneapolis, MN (US); Brett Allyn Williams, North Oaks, MN (US); William J. Drasler, Minnetonka, MN (US)

(21) Appl. No.: 17/075,409

(22) Filed: Oct. 20, 2020

(51) Int. Cl. A61N 1/37; A61N 1/05

(52) U.S. Cl. CPC A61N 1/371; A61N 1/056

(57) ABSTRACT

A pacing lead for temporary atraumatic placement via transvascular access on an endocardial surface of a heart chamber of an animal body. The pacing lead body has a curled shaft at the distal end region of the pacing lead body having a plurality of electrode sites. Each of the electrode sites is individually connected via an electrode conduction wire to a switch box, which receives generator signals from a pulse generator and directs the electrode generator signal to specific electrode sites. A push-pull element is connected to the lead body distal end. A tension-compression member connects to the push-pull element and provides tension and compression to the push-pull element.

### CROSS REFERENCE TO RELATED APPLICATIONS

This patent application refers to U.S. Pat. No. 10,773,076 entitled “Temporary Pacing Lead, issued Sep. 15, 2020, which is incorporated herein by reference.

### BACKGROUND OF THE INVENTION

Temporary pacing is performed in patients having cardiac arrhythmias as a bridge to permanent pacing or to recovery; temporary pacing also provides prophylactic utility for specific medical procedures including, for example, transcatheter aortic valve replacement (TAVR) procedures. Such arrhythmias can manifest as bradycardia or tachycardia and can result in hemodynamic instability to the patient. Often bradycardia can occur because of sinus node dysfunction or atrioventricular block. Acute therapy can be obtained via placement of a temporary lead in the right ventricle (RV); the temporary pacing lead receives an electrically generated signal from an external pulse generator located external to the patient.

Current temporary pacing leads are generally placed via a percutaneous transvenous access, via a direct epicardial placement of the electrode via a surgical access site or transcutaneous using patches placed on the body surface, i.e., skin. The pacemaker lead can be a unipolar lead with the negative or cathode electrode located at or near its distal end. Alternately, the lead can be a bipolar lead thereby containing both the negative cathode and the positive anode on the lead body separated by a small distance of a few millimeters. The unipolar lead requires that a separate anode be located adjacent the subcutaneous tissue at a remote location located several inches away from the cathode. The unipolar lead provides for a greater ease of capture of the electrical pulse by the myocardium from the pacemaker generator. The bipolar lead provides a benefit over the unipolar lead for requiring a lower threshold energy to obtain capture and hence has greater application for permanent pacing with a preserved battery life for the implanted pulse generator.

Often permanent pacing leads are implanted following TAVR procedures due to new bundle branch block or advanced aortic valve (AV) block, i.e., complete heart block with loss of the conduction signal to the ventricles of the heart. In many cases the permanent pacing lead is not needed after a period of approximately one month due to return of the conduction pathways to a normal state. Removal of such permanent pacing leads can be difficult due to attachment of the lead distal end to the surface of the heart. Often such permanent pacing leads are left in place even though they are not necessary, i.e., they do not provide a functional benefit to the patient.

Temporary pacing leads can have active fixation elements such as a distally located screw-shaped electrode that is screwed into the myocardium. or other forms of mechanical attachment. Such active fixation can hold the lead in place but is also more difficult to place during implantation. Active fixation leads carry a greater likelihood of myocardial perforation and potential for tamponade. Other temporary leads can be more easily and quickly placed without active or passive fixation elements, but still require fluoroscopy and are easily dislodged by small movements of the pacing lead in relation to the patient, which results in loss of capture of the electrical stimulus from the pacemaker generator even due to small micro-dislodgements. Temporary pacing leads can also have flow-directed balloons located near the distal end to assist with advancement of the pacing lead in the RV chamber, but remain difficult to adequately position for capture and thus require a significant amount of manipulation under fluoroscopy for optimal positioning. Flow-directed balloons are less reliable for providing a preferred location for the pacing lead.

Current temporary pacing leads often have a general linear configuration near the distal region of the lead. A slight curve can be formed into the lead to allow it to lay against the inferolateral wall of the RV. Due to the linear configuration, the distal end of the temporary lead can be traumatic to the heart wall and can protrude, penetrate, or perforate through the wall of the heart leading to potential tamponade and which can lead to death of the patient. Placement of such linearly configured leads is performed under fluoroscopic guidance (rarely ultrasound guidance) to position the lead properly against the endocardial surface of the heart and to prevent inadvertent perforation of the heart wall. Blind position carries a much higher risk of perforation and unreliable pacing capture.

Due to the general linear configuration of standard temporary leads, the distal region of the lead does not easily maintain a sustained position adjacent to the optimal endocardial surface which is needed to maintain sustained electrical capture of the myocardial tissue. Instead the distal region of the lead can easily dislodge and lose capture shortly following placement. The proximal shaft of such a linearly-configured temporary lead is often secured with multiple sutures and large, cumbersome adhesive dressing near its manifold to the patient's tissue near the access site to help prevent dislodgement of the lead and loss of capture. However patient movement and inherent motion of the heart tend to easily result in dislodgement of the lead and resultant loss of capture. If the temporary pacing lead should need to be repositioned due to loss of capture as a result of dislodgement, care must be taken and once again requires the use of fluoroscopy to ensure that the pacing lead does not perforate the myocardial tissue during repositioning. This often requires patient transfer back to the cardiac catheterization laboratory.

Vascular access is obtained via a percutaneous transvenous site through which the temporary pacing lead is advanced under fluoroscopic guidance. The lead can be provided percutaneous access using the femoral vein (FV), subclavian vein (SCV), the internal jugular vein (UV), or other suitable venous access sites. The lead is then advanced through the right atrium (RA) and into the RV. The bipolar lead has a negative electrode or cathode and an adjacent positive electrode or anode, which are found on the distal segment of the lead positioned to obtain adequate contact with the myocardium of the RV such that the electrical pulse from the pulse generator is transmitted to and captured by the myocardium. Required radiation exposure while using fluoroscopy can be detrimental to a patient.

Several complications exist during the placement and operation of temporary pacemaker leads. Such complications include myocardial damage, generation of arrhythmias, perforations of the myocardium, tamponade, trauma to the tricuspid valve, and dislocation or dislodgement of the pacing lead with loss of capture. Many of the pacer leads are traumatic and their distal end, wherein the electrodes are located, can penetrate the myocardial tissue, or perforate the atrial or ventricular wall of the heart.

What is needed is a temporary pacing lead that is easily placed and is atraumatic to the myocardial tissues of the heart including the tissues of the RA and RV. The lead should be placed without the need for fluoroscopy and its associated inconvenience, time, and radiation, also preferably without the need for echocardiographic guidance. The lead should be configured such that more than one cathode and anode is positioned on the distal lead such that positioning of the lead does not require precise visualization as required by current standard leads which are placed using fluoroscopy. The lead should not be easily dislodged once it is placed in the RV. The lead should be easily stabilized or held in a stationary position in relation to the access sheath such that dislodgements and loss of capture is reduced. If the lead is displaced, it should be easily repositioned without the need for fluoroscopy. The temporary pacing lead should be easily removed following the return of a stable patient rhythm or placement of a permanent pacemaker.

A temporary lead should be able to provide benefit to those patients who have undergone a TAVR procedure and have encountered bundle branch block. Such patients should have access to a temporary pacing lead system that can be placed for a period of over a month until it is determined whether there will be return of their normal conduction to the ventricles of the heart. The temporary pacing lead should be able to provide consistent contact with the endocardial surface of the heart for this one-month period following TAVR and still be easily removed from the heart. A permanent pacing lead should be able to be easily implanted to replace the temporary lead.

### SUMMARY OF THE INVENTION

The present invention is directed to a temporary pacing lead that overcomes the objections found in current standard temporary pacing leads. The pacing lead can be used in any of the four chambers of the heart. Most often, however, the pacing lead is placed into the RV. Hence, the discussion presented will focus on this chamber of the heart.

The present invention is specifically directed to a pacing lead for temporary atraumatic placement via transvascular access on an endocardial surface of a heart chamber of an animal body part to deliver an electrical signal comprising a lead manifold located outside the animal body; and a pacing lead body connected to the lead manifold, the pacing lead body having a proximal end and a distal end, wherein the pacing lead body comprises a curled shaft at the distal end region of said pacing lead body, wherein the curled shaft has a distal end and a proximal end and a curved shaped memory for temporary placement of the curled shaft against the endocardial surface, wherein the curled shaft further comprises a plurality of electrode sites, which electrode sites are connected via electrical continuity such that at least one of the plurality of electrode sites is adapted temporarily connect to the endocardial surface, wherein each of the plurality of electrode sites is connected to an individual conduction wire, wherein each of the plurality of conduction wires extends along the pacing lead body to connect to an individual electrode connector on the lead manifold, wherein each of the plurality of electrode connectors is individually connected via the electrode conduction wire to a plurality of electrode receptacles of a switch box, wherein the switch box is adapted to receive generator signals from a pulse generator and direct the electrode generator signal to specific electrode sites.

The present invention is further directed to a pacing lead for temporary atraumatic placement via transvascular access on an endocardial surface of a heart chamber of an animal body to deliver an electrical signal comprising a lead manifold located outside the animal body; a pacing lead body connected to the lead manifold, the pacing lead body having a proximal end and a distal end, wherein the pacing lead body comprises a curled shaft at the distal end region of said pacing lead body, wherein the curled shaft has a distal end and a proximal end and a curved shaped memory for temporary placement of the curled shaft against the endocardial surface, wherein the curled shaft further comprises a plurality of electrode sites, which electrode sites are connected via electrical continuity such that at least one of the plurality of electrode sites is adapted temporarily connect to the endocardial surface, wherein each of the plurality of electrode sites is connected to an individual conduction wire, wherein each of the plurality of conduction wires extends along the pacing lead body to connect to an individual electrode connector on the lead manifold, wherein each of the plurality of electrode connectors is individually connected via the electrode conduction wire to a plurality of electrode receptacles of a switch box, wherein the switch box is adapted to receive generator signals from a pulse generator and direct the electrode generator signal to specific electrode sites; and a push-pull element connected to the lead body distal end, wherein the push-pull element traverses external to the lead body distal region, wherein the lead body includes a control opening at the proximal of the curled shaft, wherein the push-pull element extends through the control opening into a control lumen within the lead body to the lead manifold at the proximal end of the lead body, wherein the lead manifold includes a tension-compression member for securing the push-pull element and providing tension and compression to the push-pull element.

The curled shaft of the present invention contains at least one and preferably a plurality of electrode sites all of which are connected via electrical continuity to form a single cathode or electrode which characterizes the present temporary pacer lead as a unipolar lead. For the unipolar lead, the positive electrode is located on an external surface of the body such as, for example, the patient's back. The plurality of cathodes sites allows the present unipolar temporary pacing lead to be easily placed within the chamber of the heart such that at least one of the cathodes sites is in contact with a region of the endocardium to create a capture site that is needed to temporarily pace the heart. The shapeable curled shaft applies a small outward force onto two adjacent or opposing walls of the heart chamber and hence place the cathode sites into contact with the endocardial surface of the myocardium to ensure electrical contact and capture of the pacing signal from the pulse generator.

Due to this multiplicity of electrode sites and combined with the atraumatic shape of the distal curled region, the pacer lead of the present invention can be placed without fluoroscopic imaging or possibly under echo guidance without the concern for perforation of the heart wall while ensuring that at least two of the electrode sites is contributing to electrical capture of the myocardium for temporary pacing. Placement of the pacing lead will not require the fluoroscopic guidance since the curled distal region with the multiplicity of electrode sites does not require the visualization provided by fluoroscopy as required by standard leads to reduce the likelihood for pacing lead perforations and ensure precise placement for standard temporary pacing leads. Confirmation of proper placement of the curled distal region into the RV can be guided blindly while observing electrocardiogram (EKG) heart conduction signals indicative of P wave and Q@RS wave changes in amplitude or advancing blindly while the lead is in a pacing mode and demonstrating atrial conduction complexes and transitioning to ventricular conduction complexes. Transthoracic echo may alternately be used as a default strategy to adjust the position of the lead.

This temporary pacing lead embodiment of the present invention has a unipolar cathode electrode rather than a bipolar electrode placed on the lead body. The unipolar cathode allows the present invention to provide capture of the electrical pulse signal by the myocardium easier than a bipolar electrode due to the ability to provide a larger current density required to reach a capture threshold. For temporary pacing, the ease of myocardial capture is of greater importance than the lower capture threshold found in bipolar leads than the need to conserve battery power for a permanent pacemaker. The ease of capture combined with the ability to capture with any of the multiplicity of cathode sites provides the multiple unipolar cathode sites of the present invention with an advantage over other pacing leads to provide an even greater ease and consistency of capture.

Placement of the temporary pacing lead of the present invention may be performed by first placing a placement stylet or guidewire into an internal lumen of the pacing lead. The stylet, for example, may have a linear or curved shape that does not form a closed loop; the stylet has a radius of curvature that may be much larger than the radius of curvature of the closed loop of coiled shaft of the temporary pacing lead of some embodiments of the present invention. Placement of the stylet into the lumen of the pacing lead causes the distal coiled shaft of the pacing lead to form a more gently curved shape that allows the pacing lead to traverse the venous vasculature to the heart and cross the tricuspid valve (TCV) annulus. The distal end of the pacing lead can be a closed end such that the stylet is able to extend within the internal lumen of the pacing lead but cannot extend distally beyond the closed distal end. Once the pacing lead is across the TCV, the pacing lead can be advanced into the heart chamber where the distal region of the pacing lead can form a distal curled region within the RV. The pacing lead can be advanced potentially under echo guidance to place the distal curled region into contact with the distal RV lateral wall, apex, and septal wall of the RV. The distal curled region has a radius of curvature similar to a section of the endocardial surface of the chamber of the heart and hence it confers an atraumatic character. The stylet can be retained within the lumen of the pacing lead to form a closed loop in the lead distal region; the closed loop makes contact with the walls of the RV chamber to provide optimal threshold values for capture of the pulse signal and for providing an optimally high sensed signal voltage.

In another embodiment the pacing lead can have an open distal end such that the pacing lead can pass over a floppy coiled guidewire that has been placed through the vasculature and into the right ventricle. This atraumatic guidewire would have a very soft curved distal region positioned within the chamber of the right ventricle and a stiffer and straighter shaft located within the right atrium and venous vasculature extending from the access site to the heart. The pacing lead of this embodiment can then be advanced over the wire into the right ventricle and around the coiled wire positioned in the right ventricle in a safe and atraumatic manner. In this embodiment, the temporary lead can be delivered under fluoroscopy. The lead and guidewire together form a curled shaft that is atraumatic to the endocardial surface of the RV.

To assist in placing the lead into the RV under hemodynamic guidance, a distal orifice or orifices can be placed in the distal region of the coiled shaft at a location distal to the electrode sites. The orifices connect to a fluid-filled lumen and, when connected to a pressure transducer, mimic characteristics of the chamber in which it rests. Again, observation of the pressure signal within the blood vessel or chamber via a pressure transducer that is sealingly connected to the manifold pressure port provides the operator with a distinguishing pressure that is characteristic of the location of the distal region of the pacing lead thereby giving knowledge of the location of the distal region of the pacing lead to the operator. The side or end orifices can also be used for delivery of contrast medium or for delivery of a drug to the central (intracardiac) circulation via the manifold port when it is connected, for example to a syringe.

If the pacing lead becomes dislodged later period other than the initial lead placement setting, the pacing lead can be easily and safely repositioned to regain capture possibly under hemodynamic guidance without concern for lead perforation through the heart wall. Due to the curled atraumatic distal region and the multiplicity of electrode sites, a small adjustment of the pacing lead either via distal or proximal movement of the pacing lead body will result in electrical recapture of the myocardial via any one of the electrode sites found in the curled distal region. Repositioning of the pacing lead can occur either blindly or with hemodynamic echocardiographic or, if necessary, echocardiographic or fluoroscopic guidance.

In one embodiment, an echogenic coating is applied to the pacing lead body, segments of the lead body, as well as the distal coiled region of the pacing lead. The echogenic coating can aid in visualizing the pacing lead under echo guidance during initial placement or repositioning of the pacing lead. Portable echocardiographic image can be easily performed transthoracically at the bedside.

In another embodiment the anode of the present invention is provided as a component of the introducer sheath that provides passage for the temporary pacing lead at the access site into the overlying soft tissue and vasculature of the body. The anode may also be positioned contact with adjacent soft tissue overlying the vascular entry site. This then can be electrically coupled to the temporary pulse generator. Alternately, the anode can be attached to the introducer sheath as a sticky patch electrode or a sticky flange electrode that is placed into contact with the subcutaneous tissue or skin at the access site into the venous vasculature.

Advantages are provided by a loop configuration for the coiled region of the temporary pacing lead including atraumatic contact with the myocardial wall and providing an outward force on the multiplicity of electrodes against endocardial wall segments of the heart chamber to attain consistent capture of the electrical stimulation signal. Most embodiments of the present invention are not required to have a closed loop configuration to provide atraumatic contact with the myocardium and maintain effective and stable capture. An open loop distal coil can have numerous shapes and sizes to maximize good position on multiple sites. Such other embodiments with a closed loop provided by the embodiment with different shapes and sizes give the additional advantage for removal of the pacing lead from the heart chamber without potential for entanglement and potential disruption of cordae tendineae which would create an incompetent TV. The distal region of the curled shaft that forms the curled loop is formed with a low bending modulus material such that the curled loop can be easily bent during removal of the pacing lead, further minimizing this risk.

Temporary pacing leads are often placed via the femoral vein to provide stable electrical signaling to the heart due to Brady- or Tacky-arrhythmias. The leads can also be placed prophylactically peri-MI, during TAVR procedures, etc. The femoral vein provides a reliable access site that is easy and quick to access. The standard temporary lead is not intended for long-term use; it is placed within a femoral vein introducer sheath that is inserted percutaneously into an access site vein of the body. After a few days, this access site in the groin is often subject to premature infection which may require removal of the introducer sheath and the pacing lead. Further, this access site, generally used in the cardiovascular lab, renders the patient highly immobile unlike the internal jugular or subclavian vein access.

Temporary pacing leads are often used during therapeutic procedures such as TAVR, for example. The temporary lead may be provided access into the vasculature via the internal jugular, subclavian, or femoral veins for temporary pacing during the TAVR procedure. It is commonly used for rapid ventricular pacing during valve deployment and often for one to two days and possibly longer. If a permanent pacing lead is required at the time of discharge, the permanent pacemaker system is generally implanted from a subclavian approach. Such permanent pacing leads and pulse generators are significantly more expensive.

One embodiment of the present invention is a temporary lead that is fully implantable along with the pulse generator. These leads could be coupled with a miniaturized temporary pulse generator with rudimentary function such as VVI only, for example. This is made possible via novel miniaturized circuit boards. Such a lead can be used, for example, following a TAVR procedure wherein the patient has acquired conduction system defects that requires temporary fully implantable pacing system until a follow-up date when it is determined if a permanent pacing device is needed. The temporary lead of the present invention can be placed directly into the vasculature without the use of an introducer sheath. Both the lead and its associated miniaturized pulse generator are implanted for a limited duration. The benefits of the above lead embodiments are preserved. When left in place for weeks only (range 3-8 weeks), the lead and pulse generator would be expected to be removed with low probability of complications. The present temporary lead does not require tines or other fixation mechanisms to attach the temporary pacing lead to the endocardial surface to maintain adequate contact, and basic VVI pacemaker function. This is facilitated with an RV apical coil with a multiplicity of electrodes that will automatically switch to an optimal pacing vector (or electrode pair). The fully implantable lead of the present invention is an inexpensive system based on the simplicity of the required functionality.

The objects and advantages of the invention will appear more fully from the following detailed description of the preferred embodiment of the invention made in conjunction with the accompanying drawings.

### BRIEF DESCRIPTION OF THE DRAWINGS

FIG. 1A is a partially perspective plan view of a curled temporary pacing lead with a closed loop having a multiplicity of cathode electrodes in the right ventricle of the heart.

FIG. 1B is a plan view of a curled temporary pacing lead with an open loop.

FIG. 1C is a cross-sectional top view of the introducer sheath manifold and pacing lead manifold showing a sheath indicator and lead indicator to provide the ability to appropriately orient pre-curved distal catheter.

FIG. 2 is a plan view of a temporary pacing lead showing the multiplicity of cathodes in the distal region and their connection to the cathode conduction wire.

FIG. 3A is a plan view showing the distal region of a temporary pacing lead with a small overlap portion for the coiled shaft of a closed loop with a round shape.

FIG. 3B is a plan view showing the distal region of a temporary pacing lead with a large overlap portion for the coiled shaft of a closed loop with an oval shape.

FIG. 3C is a plan view showing the distal region of a temporary pacing lead with a large overlap portion for the coiled shaft of a closed loop.

FIG. 4 is a plan view showing the distal region of a temporary pacing lead with an echogenic coating located on the outer surface of the coiled shaft.

FIG. 5 is a plan view of a temporary pacing catheter crossing the heart annulus with a stylet contained in the central lumen.

FIG. 6 is a plan view of a temporary pacing catheter entering the heart chamber over a stylet within the central lumen.

FIG. 7A is a plan view of temporary pacing lead with a stylet in the lead central lumen and extending to the distal end of the lead.

FIG. 7B is a plan view of temporary pacing lead with a stylet in the lead central lumen and having the stylet retracted to allow the lead distal region to form a curled shaft.

FIG. 8A is a partially perspective view of a temporary pacing lead with an open distal end entering the heart chamber over a guidewire with a distal floppy end.

FIG. 8B is a partially perspective view of a temporary pacing lead with an open distal end advancing over a guidewire to the heart apex.

FIG. 8C is a partially perspective view of a temporary pacing lead with an open distal end advancing over a guidewire to form a closed loop.

FIG. 9A is a plan view of a temporary pacing lead having an open distal end and showing a guidewire passing through the lead central lumen.

FIG. 9B is a plan view of a temporary pacing lead having an open distal end and showing a guidewire being retracted to allow the lead distal region to form a closed loop.

FIG. 10A is a plan view of an introducer sheath having an anode attached near the manifold, the anode is a sleeve anode.

FIG. 10B is a plan view of an introducer sheath having an anode attached near the manifold, the anode is a flange anode.

FIG. 10C is a plan view of an introducer sheath having an anode attached near the manifold, the anode is a patch anode; the introducer has a locking screw to provide friction with a pacing lead body to prevent lead migration.

FIG. 11 is a plan view of a temporary pacing lead that is a bipolar lead that has a multiplicity of both cathode electrodes and anode electrodes in the curled shaft.

FIG. 12A is a plan view of a temporary pacing lead with a curled shaft showing an open loop.

FIG. 12B is a sketch that defines the term lead equilibrium loop angle.

FIG. 12C is a plan view of a distal region of a temporary pacing lead showing a lead equilibrium loop angle of 270 degrees.

FIG. 12D is a plan view of a distal region of a temporary pacing lead showing a lead equilibrium loop angle of 90 degrees.

FIG. 12E is a plan view of a distal region of a temporary pacing lead showing a lead equilibrium loop angle of zero degrees.

FIG. 12F is a plan view of a distal region of a temporary pacing lead demonstrating the definition of a lead bending modulus.

FIG. 12G is a plan view of a distal region of a temporary pacing lead showing a lead equilibrium loop angle of 360 degrees and having an overlap portion.

FIG. 13A is a partial perspective view of a temporary pacing lead positioned within a right ventricle and having a stylet contained within the lead central lumen; the lead contacts two walls of the right ventricle.

FIG. 13B is a plan view of a straight pacing stylet with a stiffer stylet proximal region and a more flexible stylet distal region.

FIG. 13C is a sketch that defines the term lead-stylet loop angle.

FIG. 13D is a plan view of a distal region of a lead-stylet curled shaft demonstrating the definition of a lead-stylet bending modulus.

FIG. 13E is a plan view of a distal region of a stylet with a stylet curled shaft showing a stylet radius of curvature.

FIG. 13F is a sketch that defines the term stylet loop angle.

FIG. 13G is a plan view of a distal region of a stylet curled shaft demonstrating the definition of a stylet bending modulus.

FIG. 13H is a cross-sectional top view of a lead manifold and stylet manifold showing a lead indicator and stylet indicator to provide alignment of the lead and the stylet.

FIG. 14A is a partial perspective view of a temporary pacing lead located in the apex of the heart with a removal stylet inserted in the lead central lumen to assist with removal of the pacing lead.

FIG. 14B is a plan view of a proximal region, distal region, and distal tip of a removal stylet.

FIG. 15A is a plan view of a proximal region and distal region of a vascular stylet.

FIG. 15B is a plan view of a distal region of a lead equilibrium loop of a temporary pacing lead.

FIG. 15C is a plan view of the pacing lead proximal and distal region having a vascular stylet inserted into the lead central lumen.

FIG. 16A is a plan view of a ventricular placement stylet.

FIG. 16B is a plan view of a lead distal region forming a lead equilibrium loop upon partial retraction of the ventricular placement stylet.

FIG. 16C is a plan view of a ventricular placement stylet having a stylet curled shaft.

FIG. 16D is a plan view of a lead distal region having a ventricular placement stylet inserted into the lead central lumen to form a lead-stylet radius of curvature.

FIG. 17 is a partially perspective view of a temporary pacing lead entering the heart annulus and also a view of the temporary pacing lead advanced into the heart apex and having a lead loop equilibrium radius of curvature.

FIG. 18 is a partially perspective view of a temporary pacing lead located in the heart apex and having a pacing stylet inserted into the lead central lumen to enlarge the radius of curvature to a lead-stylet radius of curvature to contact two walls of the heart chamber.

FIG. 19 is a partially perspective view of a temporary pacing lead located in the heart apex and having a removal stylet inserted into the lead central lumen to assist with removal of the lead from the heart chamber, preventing sub-tricuspid valve apparatus entanglement.

FIG. 20 is a perspective view of a lead distal region having a guidewire with a spiral loop or pig tail extending of the lead open distal end and allowing the lead and guidewire to be advanced safely into the heart chamber without echo guidance or fluoroscopic guidance.

FIG. 21A is a plan view of a temporary pacing lead having a control fiber that can be activated under tension to pull the lead distal end into contact with the lead body to form a closed loop; the lead is in a linear configuration to traverse the vasculature.

FIG. 21B is a plan view of a temporary pacing lead having a control fiber that can be activated under tension to pull the lead distal end into contact with the lead body to form a closed loop; the lead has formed a closed loop for entry into the heart annulus and for removal from the heart chamber to prevent sub-tricuspid apparatus entanglement.

FIG. 21C is a plan view of a temporary pacing lead having a control fiber that can be activated under tension to pull the lead distal end into contact with the lead body to form a closed loop; the lead is in an open loop configuration suitable for pacing the heart chamber.

FIG. 21D is a perspective view of a pacing lead distal region utilizing a push-pull element.

FIG. 21E is a perspective view of the pacing lead distal region of FIG. 21D illustrating a fully curled shaft.

FIG. 21F is a perspective view of the pacing lead distal region of FIG. 21D illustrating the shaft in a complete loop.

FIG. 22A is a perspective view of the distal feature of the pacing lead illustrating the push pull element alongside the lead distal region.

FIG. 22B is a perspective view of the distal feature of the pacing lead of FIG. 22A illustrating tension on the push pull element.

FIG. 22C is a perspective view of an alternative embodiment of the distal feature of the pacing lead of FIG. 22A.

FIG. 22D is a perspective view of an alternative embodiment of the distal feature of the pacing lead of FIG. 22C illustrating a closed loop.

FIG. 22E is a perspective view of an alternative embodiment of the distal feature of the pacing lead having the push-pull element attached at or near a location near the feature base of the distal feature of the pacing lead.

FIG. 22F is a perspective view of the embodiment of FIG. 22E illustrating a closed loop.

FIG. 22G is a perspective view of the embodiment of FIG. 22E illustrating a recess in the lead body.

FIG. 23A is a perspective view of the introducer sheath illustrating a locking assembly for locking the pacing lead to the introducer sheath.

FIG. 23B is a perspective view of the introducer sheath of FIG. 23A within the arterial wall.

FIG. 23C is a perspective view of the introducer sheath of FIG. 23A illustrating an extension tube.

FIG. 23D is a perspective view of the introducer sheath of FIG. 23A illustrating a locking assembly.

FIG. 23E is a top plan view of split ring showing three segments.

FIG. 23F is a perspective view of an alternative embodiment of the locking assembly.

FIG. 24A is a perspective view of a standard single cathode unipolar pacing lead connected to the pulse generator of the present invention.

FIG. 24B is a perspective view of a standard single cathode unipolar pacing lead connected to the pulse generator of the present invention illustrating an alternative process.

FIG. 24C is a perspective view of a standard single cathode unipolar pacing lead connected to the pulse generator of the present invention illustrating a multiplicity of electrode sites.

FIG. 24D is an alternative perspective view of a standard single cathode unipolar pacing lead connected to the pulse generator of the present invention illustrating a multiplicity of electrode sites.

FIG. 24F is an alternative perspective view of a standard single cathode unipolar pacing lead connected to the pulse generator of the present invention illustrating an automated switch box.

FIG. 24G is an alternative perspective view of a standard single cathode unipolar pacing lead connected to the pulse generator of the present invention illustrating an alternative automated switch box.

FIG. 24H is a perspective view of a miniaturized automatic switch box.

FIG. 24I is a perspective view of miniaturized pulse generator.

FIG. 24J is a perspective view of miniaturized pulse generator combined with a mini generator.

FIG. 25A is front plan view illustrating a generator-switch-manifold component system in an implanted configuration in a patient.

FIG. 25B is a front plan view of an alternative embodiment of FIG. 25A.

FIG. 25C is a partial front plan view of the embodiment of FIG. 25B illustrating placement of a permanent lead.

FIG. 26A is a perspective view of an introducer sheath with a precurved distal end.

FIG. 26B is a perspective view of the introducer sheath of FIG. 26A fitted with a dilator.

FIG. 26C is a perspective view illustrating an introducer sheath with a precurved distal end introduced into the heart.

FIG. 27A is a front plan view of a patient illustrating the pacing lead of the present invention being placed in the patient body.

FIG. 27B is a front plan view illustrating lead distal end of the pacing lead entering right ventricle.

FIG. 28A is a front plan view illustrating a close-up embodiment of a redundant lead holder placed onto a patient's anterior abdomen.

FIG. 28B is a side plan view of the embodiment of FIG. 28A taken along lines 28B-28B of FIG. 28A.

FIG. 29A is a front plan view illustrating an alternate configuration of the redundant lead holder of FIG. 28A.

FIG. 29B is a side plan view of the embodiment of FIG. 29A.

FIG. 30A is a front plan view illustrating the placement of a temporary lead via the internal jugular vein.

FIG. 30B is a front plan view illustrating the placement of a temporary lead via the subclavian vein.

### DETAILED DESCRIPTION OF THE INVENTION

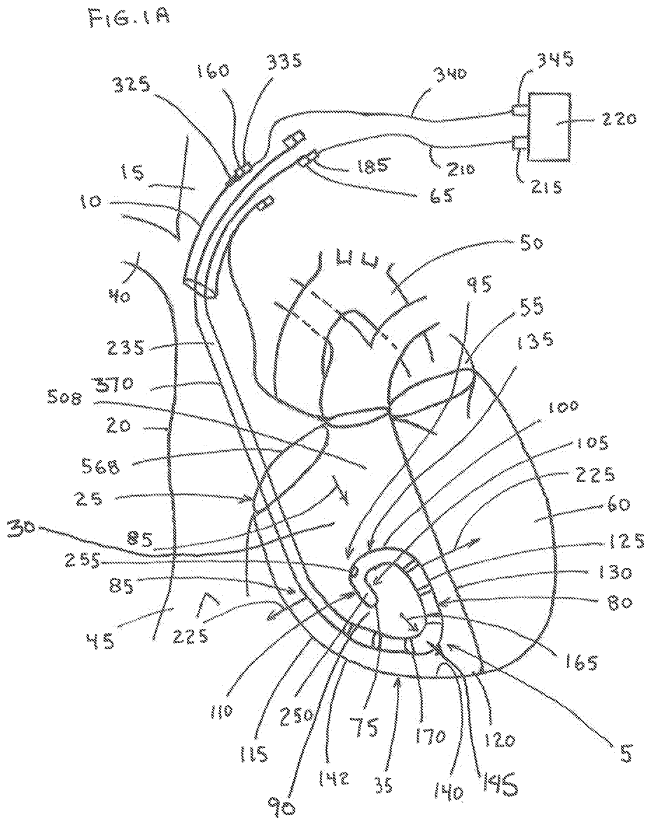

FIG. 1A shows an embodiment of the present invention for a temporary pacing lead 5. In this embodiment the pacing lead 5 is introduced through an introducer sheath 10 placed in the internal jugular vein (IJV) 15; the pacing lead 5 extends through the right atrium (RA) 20 and across the tricuspid valve (TCV) 25 and into the right ventricle (RV) 30 of the heart 35. It is understood, however, that this invention applies also to other entry sites into the vasculature including the subclavian vein (SCV) 40, the femoral vein (FV) 45, the aorta 50, or other blood vessels or conduits of the body; the pacing lead 5 can also be used to pace the RA 20, left atrium (LA) 55, left ventricle (LV) 60 or other chambers of the heart 35 or body.

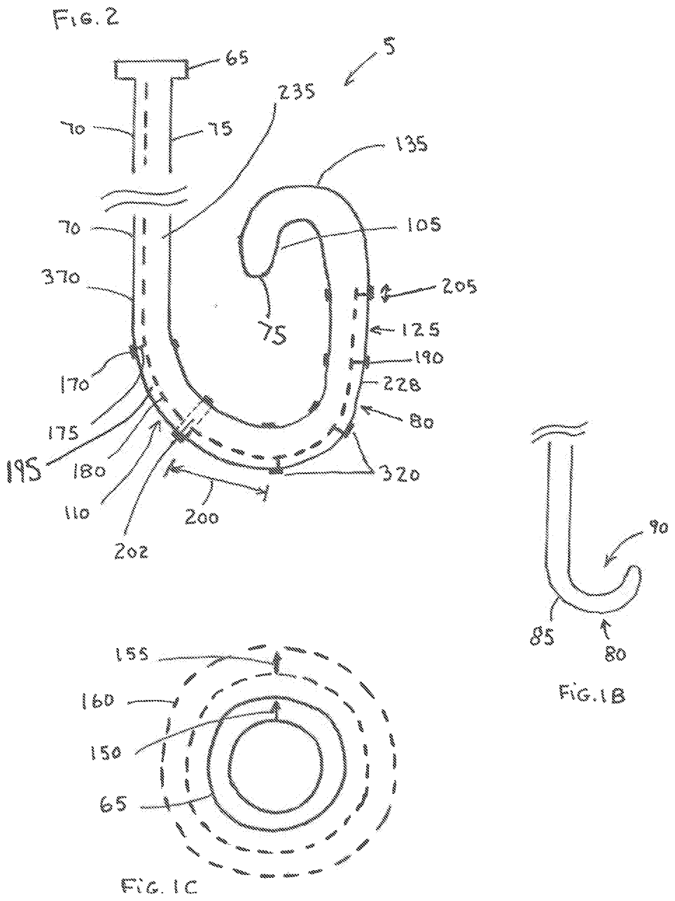

As shown in FIGS. 1A and 2 the pacing lead 5 has a lead manifold 65 located outside of the body, the lead manifold 65 is attached to the lead body 70 which extends distally through the introducer sheath 10. The lead body 70 of the pacing lead 5 extends distally to the lead distal end 75. A curled shaft 80 is formed in the lead distal region 85 of the pacing lead 5. The curled shaft 80 can have an open loop 90 as shown in FIG. 1B or the curled shaft 80 can have a closed loop 95 as shown in FIGS. 1A and 2 (and which can be referred to as a coiled shaft 100, a subgroup of a curled shaft 80). All structures for the coiled shaft 100 are understood to be included into the broader description of a curled shaft 80, and hence reference names and reference numerals used to describe a coiled shaft 100 apply to a curled shaft 80 which includes other embodiments of the present invention described subsequently in the specification.

### Curled Shaft 80 and Coiled Shaft 100

The curled shaft 80 comprised of a coiled shaft 100 has been positioned within the RV 30 and is ready for pacing of the RV 30 as shown in FIG. 1A. The coiled shaft 100 forms a closed loop 95 such that there is an overlap portion 105 that overlaps or extends adjacent to and in the same direction as another portion of the coiled shaft 100 to form a coiled or spiral shape and which is referred to herein as a closed loop 95. The curled shaft 80 has a distally directed portion 110 that follows along the lateral wall 115 of the RV 30, for example, and bends to contact or subtend the apex 120 of the heart 35, a proximally directed portion 125 that follows along the septal wall 130 of the RV 30, for example. A closing portion 135 and an overlap portion 105 completes the closed loop 95 of the coiled shaft 100 by coming into near proximity or contact with the distally directed portion 110; the lead distal end 75 does not extend outward into contact with the endocardial surface 140 of the myocardial tissue 142 of the heart 35. Each portion of the coiled shaft 100 extends in a single plane as it forms its coiled or spiral shape. The lead distal end 75 of the pacing lead 5 of this embodiment is located in the overlap portion 105 of the coiled shaft 100 which extends with a radius of curvature that is less than the distally directed portion 110 and does not contact the endocardial surface 140. At least some of the overlap portion 105 extends with a distal direction 145 like the distally directed portion 110 to form the closed loop 95. As shown in FIG. 1A the curled shaft 80 can be oriented within the RV 30 chamber such that the plane of the curled shaft 80 extends from the septal wall 130 to the lateral wall 115 or at any other orientation within the ventricular chamber. Orientation can be obtained by positioning a lead indicator 150 located on the lead manifold relative to a sheath indicator 155 located on the sheath manifold 160, for example, as shown in FIG. 1C.

### Materials Forming Lead Body 70

The pacing lead body 70 and curled shaft 80 are formed from materials found in existing pacing leads known to the art. An insulative polymer tubing formed from polyurethane or silicone, for example, can be used to form the lead body 70 and curled shaft 80 and retain the curled shape of the curled shaft 80. The profile diameter of the insulative polymer tubing and for the curled shaft 80 is preferably 5 French (Fr) with a range of 4 Fr to 8 Fr. A shaped metal wire can be embedded within the wall of the tubing to assist in forming the curled shape of the curled shaft 80. The shaped metal wire can be formed from Nitinol, Elgiloy, or other elastic material that can help retain the shape of the curled shaft 80. Nitinol (an acronym for Nickel Titanium Naval Ordnance Laboratory) is a family of intermetallic materials, which contain a nearly equal mixture of nickel (55 wt. %) and titanium. Other elements can be added to adjust or “tune” the material properties. These materials are known to exhibit unique behavior, specifically, a well-defined “shape memory” and super elasticity. The curled shaft 80 has a curled shaft radius of curvature 165 of preferably 1-2 cm with a range of about 0.5-3 cm, such that it can traverse the TCV 25 and enter the RA 20 and matches the shape of the apex 120 and mid-cavity of the chamber of the heart 35. The curled shaft radius of curvature 165 for the curled shaft 80 may be larger in the distally directed portion 110 than the proximally directed portion 125; the radius of curvature may become smaller as the curled shaft 80 extends from the distally directed portion 110 to the distal end 75 of the pacing lead 5.

### Cathode Sites 170

Located along the curled shaft 80 is a plurality of cathode sites 170 which have electrical continuity with each other; each electrode site is connected electrically via a cathode continuity member 175 to a cathode conduction wire 180 which extends along the pacing lead body 70 to the cathode connector 185 located on the lead manifold 65. Each cathode site 170 can be formed by a ring electrode 190, for example, which is placed around the insulative tubing 195 encircling the curled shaft 80. The ring electrode 190 can be formed from platinum or other metal or metal alloy used to form pacing lead electrodes. The cathode conduction wire 180 can be formed from multi-filer metal coiled wire used in current pacing leads to transmit electrical signals through the lead body 70 and curled shaft 80 to each of the ring electrodes 190 located in the distal region 85 of the pacing lead 5. Construction material for the cathode conduction wire 180 can be of a metal or metal alloy used in pacing leads currently found in the industry. The multiplicity of cathode sites 170 forms a single cathode electrode or cathode 320. The number of cathode sites 170 can range from 2 to 20 and can be located along the distally directed portion 110, the proximally directed portion 125, or other portions of the curled shaft 80. The cathode site spacing 200 between each of the cathode sites 170 or between each ring electrode 190 is enough to ensure that at least one cathode site or ring electrode 190 is able to make contact with the endocardial surface 140 such that capture of the signal from the ring electrode 190 is obtained. The cathode site spacing 200 is set at a distance of preferably 1 cm with a range of 0.5 cm-2 cm. The electrode area 202 of each ring electrode 190 or cathode site is such to provide a current density from the ring electrode 190 to the myocardium that will generate capture of the myocardium. The ring electrode length 205 for each ring electrode is preferably 3 mm with a range of 1 mm-5 mm.

### Pulse Generator 220

The cathode connector 185 located on the lead manifold 65 is connected via a cathode connecting wire 210 to the negative pole 215 of a pulse generator 220. The pulse generator 220 provides the voltage and current to the cathode electrode or cathode 320 found in the curled shaft 80 to provide temporary pacing for the patient. Standard pacing currents and voltages are used with the present invention as with standard pacing leads; adjustments can be made to the current to account for appropriate current density found for the multiplicity of cathode sites 170 to obtain appropriate myocardial capture of the electrical signal. When a specified current or voltage is delivered to the cathode 320, the signal is received by the endocardial surface 140 of the myocardial tissue 142 and the electrical signal is transmitted through the myocardial tissue 142; the signal from the cathode 320 has then been captured by the myocardial tissue 142.

Due to the multiplicity of cathode sites 170 contact of any one of the cathode sites 170 with the endocardial surface 140 can result in capture of the electrical signal from the pulse generator 220. The multiplicity of cathode sites 170 allows the pacing lead 5 of the present invention to be positioned more easily within the chamber of the heart 35 since any one of the cathode sites 170 can effectively cause capture to occur.

The coiled shaft 100 located in the lead distal region 85 of one embodiment forms a closed loop 95 that is atraumatic to the patient and will not allow the distal end 75 of the pacing lead 5 to perforate the myocardial tissue 142 since the lead distal end 75 of this embodiment is not placed into contact with the myocardial tissue 142 as found in most of the current standard pacing leads. This atraumatic shape for the distal region 85 combined with the multiplicity of cathodes sites 170 allows the pacing lead 5 to be placed without fluoroscopic guidance or echo guidance due to the atraumatic shape of the distal region 85. The present pacing lead 5 ensures a successful capture since the instant pacing lead 5 can obtain capture via any one of the multiplicities of cathode sites 170. Echo guidance may be used primarily to assist with placement and assess lead positioning. Fluoroscopy is required for placement of present standard pacing leads to ensure that the pacing lead 5 lies along the endocardial surface 140 without perforation and for more precise positioning to obtain capture.

The curled shaft 80 found in the distal region 85 of the pacing lead 5 of the present embodiment also helps to provide an outward curled shaft applied force 225 to place the cathode sites 170 into intimate contact with the endocardial surface 140 of the heart 35. The distally directed portion 110 of the curled shaft 80 and the proximally directed portion 125 of the curled shaft 80 helps to place an equal and opposite outward lead curled shaft applied force 225 onto two opposing walls of the RV chamber of the heart 35. The outward curled shaft applied force 225 pushing the curled shaft 80 against the endocardial tissues helps to ensure capture of one of the cathodes sites 170 with the myocardium and prevent dislodgement of the cathode site 170 from their lodging adjacent to the myocardial tissue 142.

In the circumstance that the cathode site 170 becomes dislodged at a later time period following the insertion of the temporary pacing lead 5, the pacing lead 5 of the present invention is easily repositioned without the need for fluoroscopy and also without the need for echo guidance. Due to the curled-shaped distal region 85 and the multiplicity of cathode sites 170, a small advancement of the lead body 70 in a distal direction 145 or retraction proximally will allow the previously captured cathode site 170 or a new neighboring cathode site 170 (or second cathode site) to make contact with the endocardial surface 140 and regain capture.

### Shapes of the Curled Shaft 80

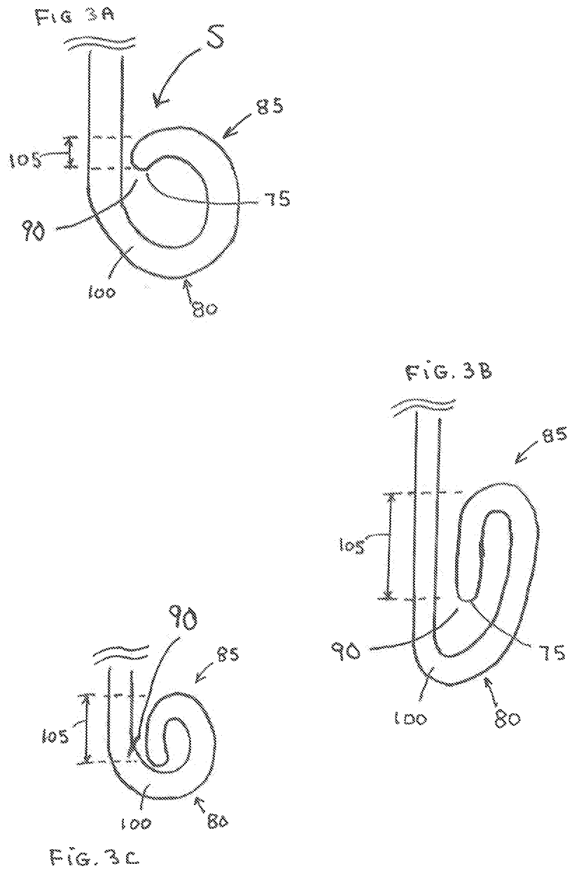

Various shapes for the curled shaft 80 have been contemplated; the curled shaft 80 can form a shape that approximates the internal endocardial surface 140 of the heart chamber 508. As shown in FIGS. 3A-3C, the curled shaft 80 can be a coiled shaft 100; the amount of overlap portion 105 can involve only the distal end 75 of the pacing lead 5 as shown in FIG. 3A or the overlap portion 105 can include a spiral that continues at a smaller radius of curvature as shown in FIG. 3C. The curled shaft 80 can be oval shaped as shown in FIG. 3B or can be more rounded as shown in FIG. 3C. The curled shaft 80 can also be formed without an overlap portion 105 as discussed in later embodiments.

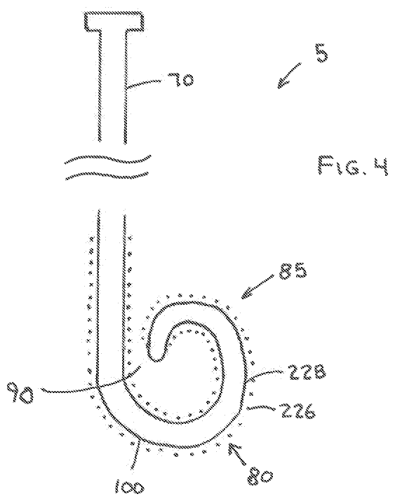

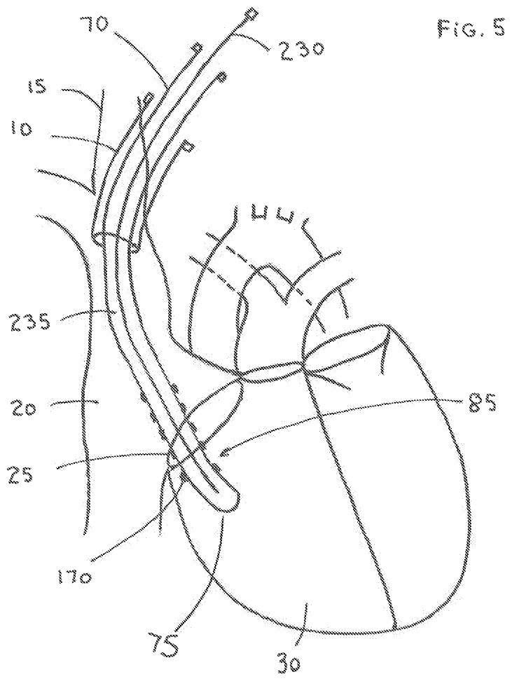

Echogenic coating 226 can be applied to the outer surface 228 of the lead body 70 and lead distal region 85 as shown in FIG. 4 to enhance its ability to be visualized on echo guidance. For example, microspheres can be adhered to or embedded into the outer surface 228 of the pacing lead 5 to reflect sound waves and enhance visualization. Alternately, echogenic materials that absorb, reflect, or generate sound waves can be located on the outer surface 228 of the pacing lead body 70 or lead distal region 85.

### Placement of Temporary Pacing Lead 5

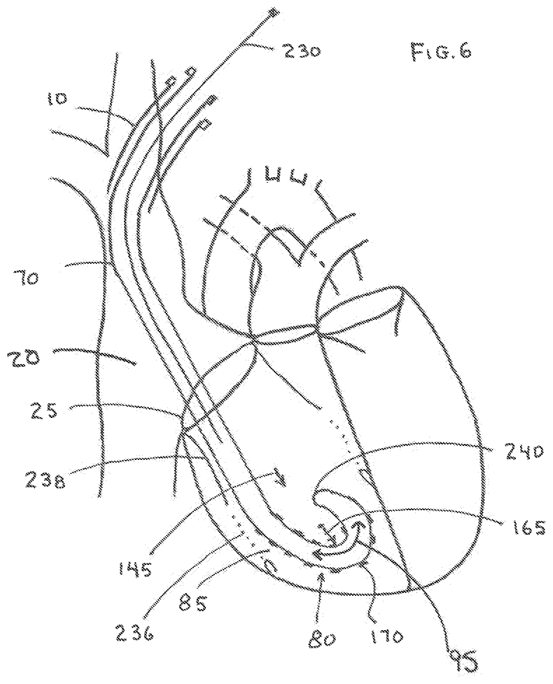

Placement of the temporary pacing lead 5 of the present invention under echo guidance or without echo guidance is shown in FIGS. 5 and 6 with the final placement as shown in FIG. 1A. As seen in FIG. 5, a linearly shaped wire or probe such as a stylet 230, for example, is slidingly placed within the lead central lumen 235 to cause the lead distal region 85 to form a generally linear shape. The radius of curvature for the distal region 85 with the stylet 230 inserted is greater than 10 cm. The stylet 230 is formed from a metal such as stainless steel, Nitinol, or other metal or composite and has a linear shape with a radius of curvature greater than 10 cm. The pacing lead 5 with the stylet 230 inserted is advanced through the introducer sheath 10 located in the internal jugular vein, IJV 15 along with the stylet 230 and across the tricuspid valve 25. A transthoracic echo (TTE) probe can be used to view the passage of the pacing lead 5 and stylet 230 across the TCV 25 as shown in FIG. 5.

While holding the stylet 230 in a fixed position, the pacing lead 5 is advanced distally into the right ventricle, RV 30. With the stylet 230 no longer located in the distal region 85, the distal region 85 initiates the formation of a curled shaft 80 that extends into the RV as shown in FIG. 6. The curled shaft 80 has a curled shaft radius of curvature of preferably 1 cm with a range of 0.5-3.0 cm to fit within the TCV 25 and not snag the cordae tendineae 236 of the heart valve 238. The lead distal end 75 of this embodiment has a closed distal end 240 that is a blunt rounded surface with no lead central lumen 235 extending therethrough and does not allow the stylet 230 or a stiff guidewire 290 to extend out of the lead distal end 75. Further advancement of the pacing lead 5 while holding the stylet 230 at a fixed position allows the lead distal region 85 to form an equilibrium configuration of a closed loop 95 as shown in the coiled region of FIG. 1A or an open loop 90 as shown in FIG. 1B.

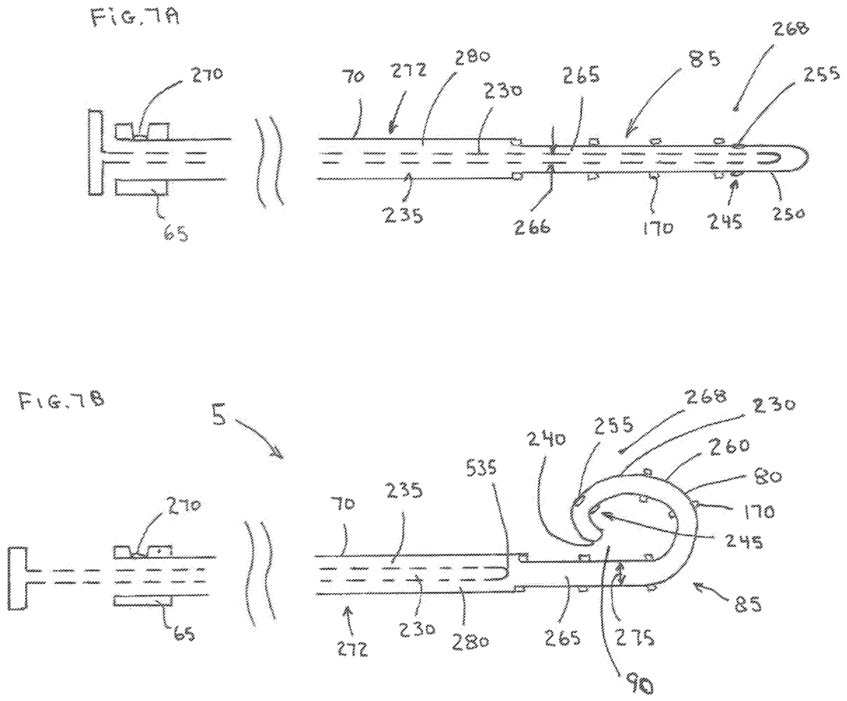

FIGS. 7A and 7B show an embodiment for the pacing lead 5 of the present invention having one or more orifices 245 located in the distal region 85 of the curled shaft 80, i.e., between the lead distal tip 250 and the distal region 85 not containing the cathode sites 170. The orifices 245 can be one or more side orifices 255 as shown in FIGS. 7A and 7B which extends through the curled shaft wall 260 and is in direct fluid communication with the lead distal lumen 265. The distal lumen 265 forms a portion of the central lumen 235 that extends throughout the lead body 70 of the pacing lead 5. The one or more orifices 245 should be smaller than the stylet diameter 266 to contain the stylet 230 within the distal lumen 265 but should provide a hydraulic diameter, i.e., a calculated diameter that is equivalent hydraulically to the distal lumen diameter 275, that does not dampen a pressure signal from the blood from an outside region 268 outside of the curled shaft 80 to the distal lumen 265 of the pacing lead 5. A manifold port 270 located on the lead manifold 65 is also in direct fluid communication with the lead central lumen 235 of the lead body 70. A pressure transducer (not shown) can thereby be sealingly connected to the manifold port 270 and detect a pressure signal from the body fluid or blood that is located on the outside of the curled shaft 80 in the lead distal region 85 of the catheter body adjacent to the side orifice 255. The operator can use the characteristic of the pressure signal which varies from the RA 20 to the RV 30, for example, to determine the location of the lead distal region 85 while the pacing lead 5 is being delivered through the vasculature and into the heart chambers 508. Identification can be made by the operator that the lead distal region 85 has been delivered to the RV 30. Thus, the pacing lead 5 can be delivered to the RV 30 without using fluoroscopy if desired, and can be delivered under echo guidance, if desired, with the added assistance of observing the pressure signal that is characteristic of the desired delivery location for the distal region 85 within the heart 35 and vasculature. This establishes the opportunity to deliver the lead under hemodynamic guidance.

The location of the side orifice 255 or orifices 245 should be distal to the cathode sites 170 such that the pressure signal that is received from the operator indicates the pressure of the chamber into which the operator is entering, such as the RA 20 or RV 30, for example. Also, as shown in FIGS. 1A and 7B, the side orifice 255 or other pressure sensing orifices 245 should be located in the lead distal tip 250 such that as the curled shaft 80 is located in the ventricular chamber such as the RV 80, the orifices 245 are positioned closer to the right atrium 20 and closer to the lead proximal region 272 than are the cathode sites 170 positioned to ensure that the cathode sites 170 are correctly and safely located near the apex 120 of the heart 35.

As shown in FIG. 7A, the stylet 230 can be placed within the lead central lumen 235 to allow ease of delivery of the pacing lead 5 through the vasculature as described in FIGS. 5 and 6. During the delivery of the pacing lead 5 and after achieving a final location for the pacing lead 5 within the RV 30, the operator can partially withdraw the stylet 230 out of the lead distal region 85. The distal lumen 265 then becomes available for pressure signal transmission back to the manifold port 270. The distal lumen diameter 275 needed to deliver the pressure signal without degradation or dampening of the pressure signal intensity is preferably 0.020 inches with a range 0.016-0.030 inches. The stylet 230 can reside in the proximal lumen 280 of the lead body 70 during pressure signal transmission; the proximal lumen 280 is a portion of the central lumen 235 that resides within the lead proximal region 272 of the lead body 70 and is located proximal to the curled shaft 80. The annular space between the lead body 70 and the stylet 230 must provide a hydraulic diameter (i.e., a calculated diameter that is equivalent hydraulically to the distal lumen diameter 275) that is equal or greater than the distal lumen diameter 275. Alternately, the stylet 230 can be pulled back out of the central lumen 235 such that the stylet 230 does not provide any reduction in area in the central lumen 235 that could be used for pressure signal transmission; such reduction in central lumen area could result dampening of the pressure signal that is being transmitted from the side orifice 255 to the manifold port 270.

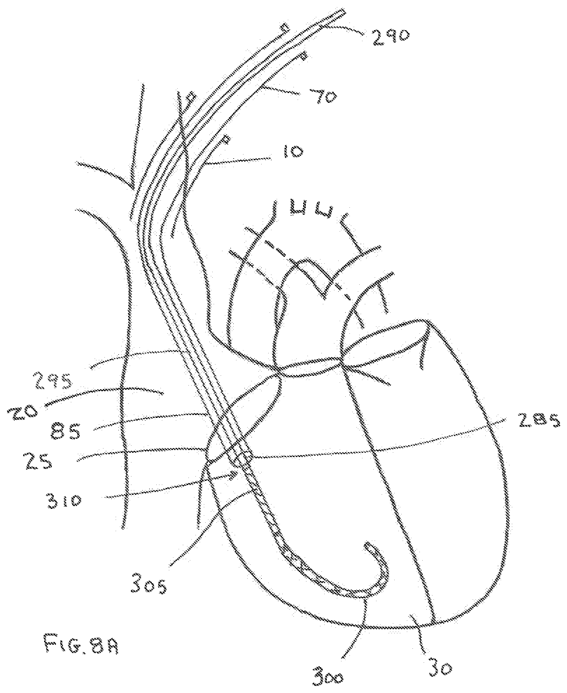

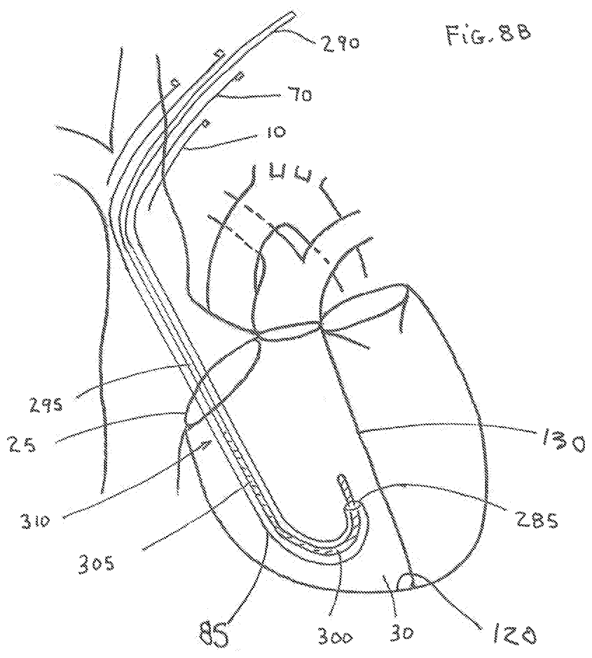

### Alternate Embodiment—FIGS. 8A-8C

An alternate embodiment for the pacing lead 5 of the present invention has an open distal end 285 as shown in FIGS. 8A-8C. As shown in FIG. 8A a stylet 230 such as a guidewire 290, for example, is first placed through the introducer sheath 10 and into the RV 30. The guidewire 290 can have a guidewire stiff region 295 that is located within the introducer sheath 10 and extending across the TCV 25. The guidewire stiff region 295 is intended to interface with the lead distal region 85 during delivery of the pacing lead 5 through the vasculature such that the lead distal region 85 becomes more linear or less curved, i.e., less curled, and can traverse the vasculature more easily to reach the RV of the heart 35. A softer guidewire curled region 300 is located in the right ventricle, RV 30 and provides an atraumatic shape that is similar to the shape of the chamber walls and apex 120 of the RV 30. Between the guidewire stiff region 295 and the guidewire curled region 300 is a guidewire transition region 305 that is intermediate both in shape and stiffness between the guidewire transition region 305 and the guidewire curled region 300; the guidewire transition region 305 has more curvature than the guidewire stiff region 295 and less curvature than the guidewire curled region 300; the guidewire transition region 305 is softer than the guidewire stiff region 295 and stiffer than the guidewire curled region 300. As shown in FIG. 8A, the pacing lead 5 has been advanced over the guidewire 290 such that the open distal end 285 of the pacing lead 5 is located across the TCV 25 at a location near the guidewire junction 310 of the guidewire stiff region 295 and guidewire transition region 305.

As shown in FIG. 8B the guidewire 290 is held in a fixed position in space as the pacing lead 5 is advanced over the guidewire 290 into the RV 30, around the apex 120 of the RV 30, and directed proximally upward adjacent the septal wall 130; the guidewire 290, as shown, is extending beyond the open distal end 285 of the pacing lead 5. Further advancement of the pacing lead 5 is shown in FIG. 8C with the guidewire 290 held fixed in position. The pacing lead 5 forms a curled distal region 85 due to its preformed curled shape and can form an overlap portion 105 of a closed loop 95. The guidewire 290 can then be removed from the pacing lead 5 prior to activation of the pacing lead 5 or alternately can remain in place within the lead central lumen 235. Various guidewire 290 shapes and lengths can be used without deviating from the present invention. For example, the guidewire 290 can have a complete coiled loop that overlaps with other portions of the guidewire 290; alternately a smaller length guidewire 290 that extends only a few centimeters into the RV can be used to deliver the pacing lead 5 of the present invention to the RV.

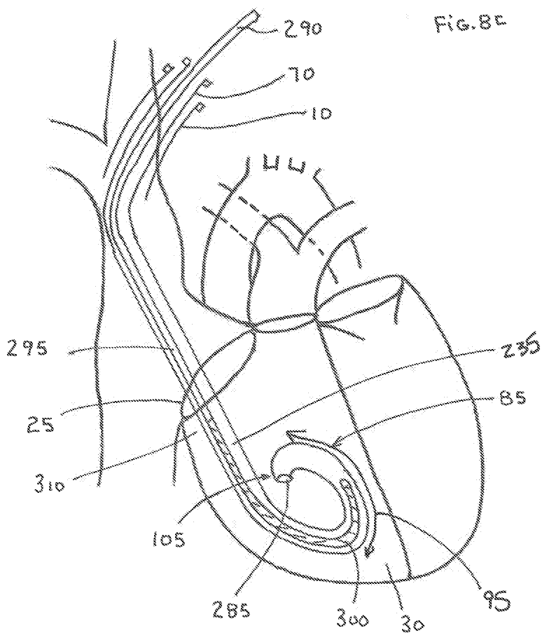

As shown in FIGS. 9A and 9B the open distal end 285 of the pacing lead 5 can provide an end orifice 315 that allows monitoring of blood pressure outside of the curled shaft 80 and within the vasculature of the body or heart 35, or an orifice for delivery of contrast medium or drugs to a region outside of the curled shaft 80 such as the vasculature or heart chambers 508. The open distal end 285 is in direct fluid communication with the manifold port 270 located on the lead manifold 65. The pacing lead 5 can be delivered through the vasculature and into one or more chambers of the heart 35 over a guidewire 290 that extends through the lead body 70 from the lead manifold 65, through the central lumen 235, and out of the open distal end 285 of the pacing lead 5. To deliver contrast medium or obtain a pressure signal from an outside region 268 outside of the curled shaft 80 adjacent to the open distal end 285 of the pacing lead 5, the guidewire 290 can be pulled back such that it is located in the proximal lumen 280 of the pacing lead 5. The distal lumen 265 of the pacing lead 5 should have a distal lumen diameter 275 of preferably 0.020 inches with a range of 0.016-0.030 inches to obtain an undamped pressure signal from the open distal end 285 to the manifold port 270. The hydraulic diameter of the proximal lumen 280 should be similar to the distal lumen diameter 275. Alternately, the guidewire 290 can be removed entirely from the central lumen 235 for delivery of contrast medium, delivery of drugs, or for measurement of pressure within a heart chamber 508 or within the vasculature adjoining the heart 35.

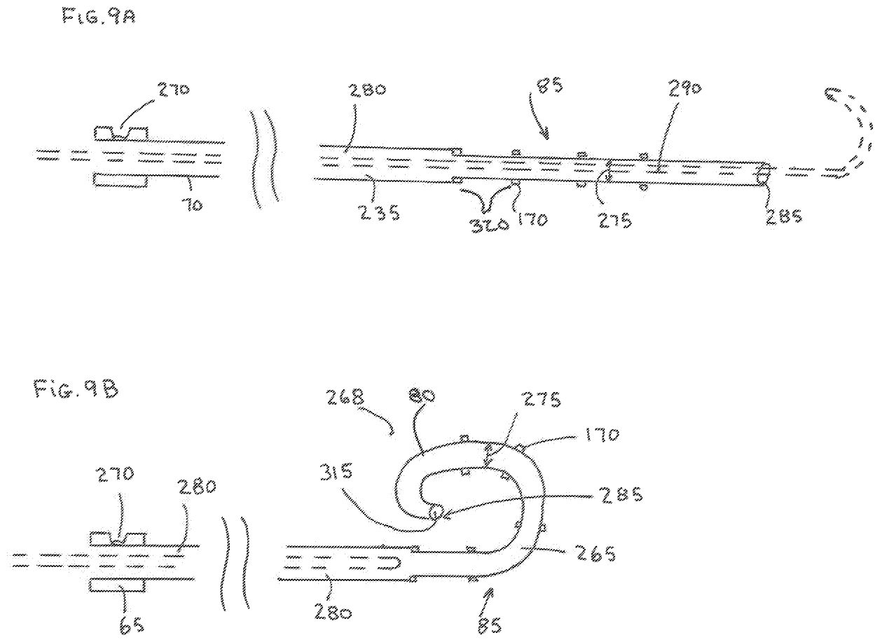

The unipolar temporary pacing lead 5 of the present invention has a cathode 320 comprised of cathode sites 170 located within the pacing lead distal region 85. In further embodiments the anode 325 is located as a component of the introducer sheath 10 as shown in FIGS. 10A-10C. In FIG. 10A the anode 325 is a sleeve anode 330 formed from a metal film that is located around a portion of the introducer sheath 10 that is near the sheath manifold 160 and is in contact with the subcutaneous tissue, the tissue tract, or the vasculature in which the introducer sheath 10 is inserted. The sleeve anode 330 is attached to an anode connector 335 located on the sheath manifold 160. The anode connector 335 is connectable via an anode connecting wire 340 to the positive pole 345 of a pulse generator 220 as shown in FIG. 1A. The sleeve anode 330 can be formed from platinum, silver, or other metal or metal alloys that provide for efficient electrical signal transmission. As shown in FIG. 10B the anode 325 can be a flange anode 350 that is joined or attached to the introducer sheath 10 and forms an electrical continuity with an anode connector 335 located on the sheath manifold 160. The flange anode 350 can be formed from standard metals used to transmit electrical signals; the flange electrode is attached to the subcutaneous tissue via adhesive that is conductive. Another embodiment for the anode 325 is shown in FIG. 10C; a patch anode 355 is electrically coupled to the anode connector 335 located on the sheath manifold 160. The patch anode 355 is attached to the subcutaneous tissue near the access site; the patch electrode is formed from materials similar to those described for the flange electrode. Located on the introducer sheath 10 of the present invention and shown in FIG. 10C is a locking screw 360 that is rotationally activated by the operator to apply a frictional force of a locking plate 365 into direct contact with a pacing lead body 70 that would pass within the introducer sheath. The frictional force between the locking plate 365 and the pacing lead body 70 would prevent inadvertent movement of the pacing lead 5 relative to the introducer sheath 10 thereby reducing the likelihood of loss of capture by a cathode site 170 of the pacing lead 5 with the endocardial surface 140 due to movement of the pacing lead body 70. Other mechanical mechanisms are anticipated to apply a frictional force from the locking plate 365 to the lead body 70 to prevent movement of the pacing lead body 70 relative to the introducer sheath.

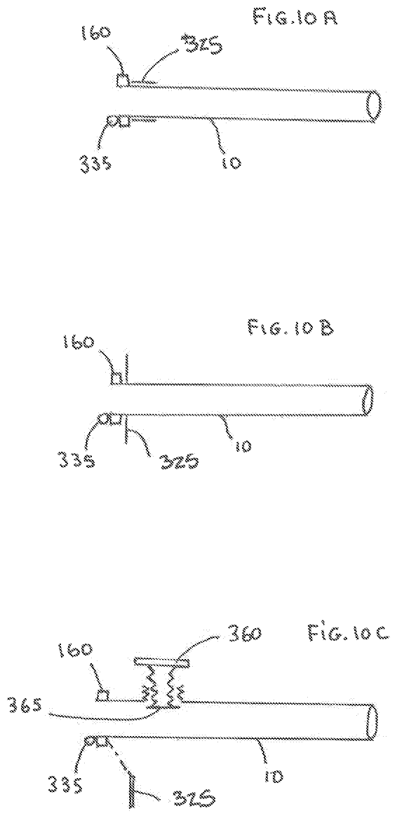

In a further alternate embodiment for the present pacing leads having a curled shaft 80, the cathode sites 170 that have been presented in earlier embodiments of the temporary unipolar pacing lead 370 can instead consist of alternating cathode sites 170 and anode sites 380 thereby transforming the unipolar pacing lead 370 of FIG. 2 into a bipolar pacing lead 375 as shown in FIG. 11. Each anode site 380 of the bipolar lead 375 of this embodiment is connected electrically via an anode continuity member 385 to the anode conduction wire 390 that extends via its own electrically insulated path through the lead body 70 to the lead manifold 65 where the anode conduction wire 390 forms an electrical continuity with an anode connector 335. The anode connector 335 is connectable to an anode conduction wire 390 that can be connected to the positive pole 345 (see FIG. 1A) of the pulse generator 220. The cathode site 170 is electrically connected to the cathode conduction wire 180 as described in earlier embodiments. The cathode sites 170 and the cathode conduction wire 180 are each electrically insulated from the anode sites 380 and the anode conduction wire 390. The bipolar pacing lead 375 of this embodiment has a similar curled shaft 80 located in the distal region 85 that is similar to the curled shaft 80 that has been discussed for the unipolar pacing lead 370 shown in FIG. 1A. The anode-cathode site spacing 395 between an anode site 380 and a neighboring cathode site 170 is similar to that provided in current standard pacing leads in order to provide current density to obtain and maintain capture; the anode-cathode site spacing 395 is 1 cm (range 0.5 cm to 2 cm). The paired site distance 405 between the anode-cathode paired sites 400 and a neighboring anode-cathode paired site 400 is 1 cm (range 0.5 cm to 2 cm) and is close enough such that small movements of the pacing lead 5 will allow one anode 325 and one neighboring cathode 320 to become an anode-cathode paired site 400. The coiled shaft 100 plus the multiplicity of anode-cathode paired sites 400 located along the coiled shaft 100 would confer both safety to the pacing lead 5 due to the atraumatic coiled shape of the distal region 85 as well as ease of forming a capture of the myocardium by at least one anode-cathode paired site 400.

It is further understood that each anode site 380 can be connected to a specific anode conduction wire 390 that extends to a specific anode connector 335 located on the lead manifold 65; thus the lead would contain a multiplicity of anode connectors 335 that are electrically insulated from each other and individually connectable to a multiplicity of anode connecting wires 340 to the pulse generator 220. Similarly each cathode site 170 can be connected to a specific cathode conduction wire 180 that extends to a specific cathode connector 185 located on the lead manifold 65; thus the lead would contain a multiplicity of cathode connectors 185 that are electrically insulated from each other and individually connectable to a multiplicity of cathode connecting wires 210 to the pulse generator 220. The pulse generator 220 is able to use an individual anode-cathode paired site 400 to detect a proper location for delivery of a temporary pacing signal. An individual anode-cathode paired site 400 located on the curled shaft 80 could then be activated by the pulse generator 220 in a specific region of the heart chamber 508 that is suitable for temporary pacing in a manner that obviates a potential for diaphragmatic capture, for example.

The previous embodiments of the present invention have shown a curled shaft 80 in a configuration of a coiled shaft 100 that has formed a closed loop 95 with an overlap region and hence the curled shaft 80 of some embodiments can have a coiled shaft 100. Embodiments of the present invention are not required to have a lead closed loop 95 forming an overlap portion 105 extending from the lead distal end 75 to the distal region 85 of a curled shaft 80. Embodiments that do not have a closed loop 95 may instead have an open loop 90 in a lead curled shaft 80 of the lead distal region 85. The lead open loop 90 provides such embodiments with an improved capability to remove the lead curled shaft 80 from the heart chamber 508 following the temporary pacing procedure without snagging and potentially tearing a cordae tendineae 236 of a heart valve 238. The embodiments having the open loop 90 also can be introduced into the chamber of the heart 35 in an atraumatic manner that does not injure the endocardial surface 140 of the heart chamber 508. The embodiments of the temporary pacing lead 5 having an open loop 90 are intended to contain the multiple cathode electrodes 170 or anode electrodes 325 as described in the previous embodiments, the electrodes can be unipolar electrodes or bipolar electrodes as described in earlier embodiments. Additionally, the pacing lead 5 is configured as described in previous embodiments to measure blood pressure via a side orifice 255 or end orifice 315 to detect and identify the chamber of the heart in which the lead distal tip 250 resides. The pacing lead 5 having an open loop 90 can have a closed distal end 240 as shown in some embodiments, or the pacing lead 5 can have an open distal end 285 as described other embodiments such as those shown in FIGS. 8A-8C and 9A-9B.

### Lead in a Lead Equilibrium Configuration

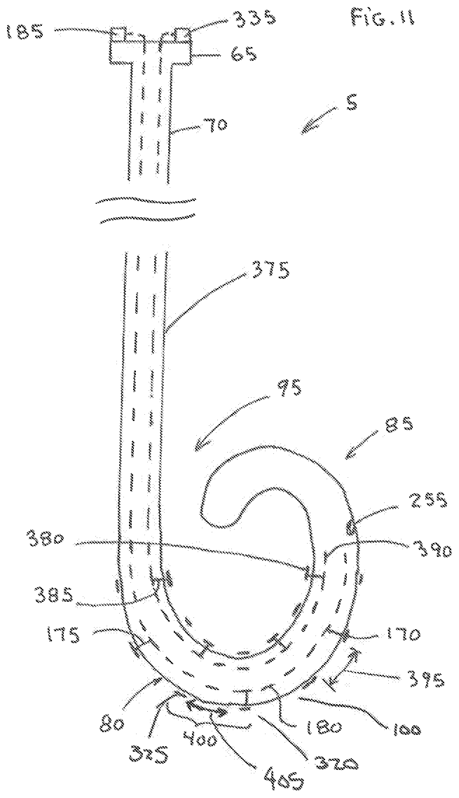

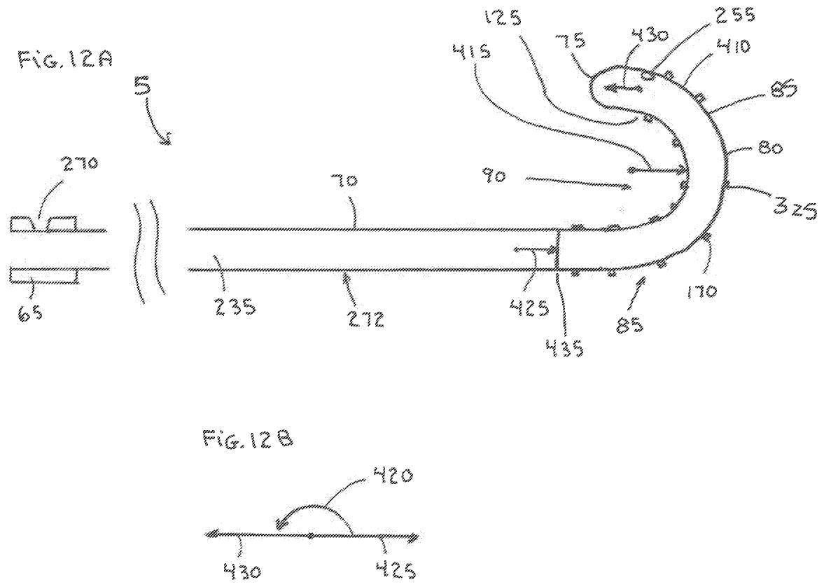

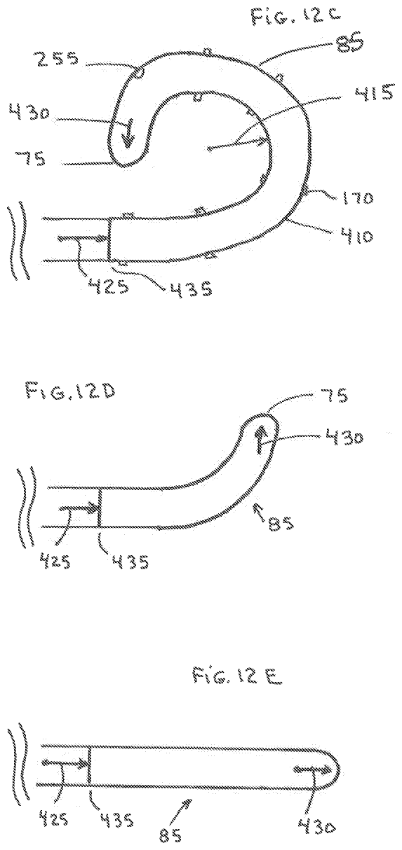

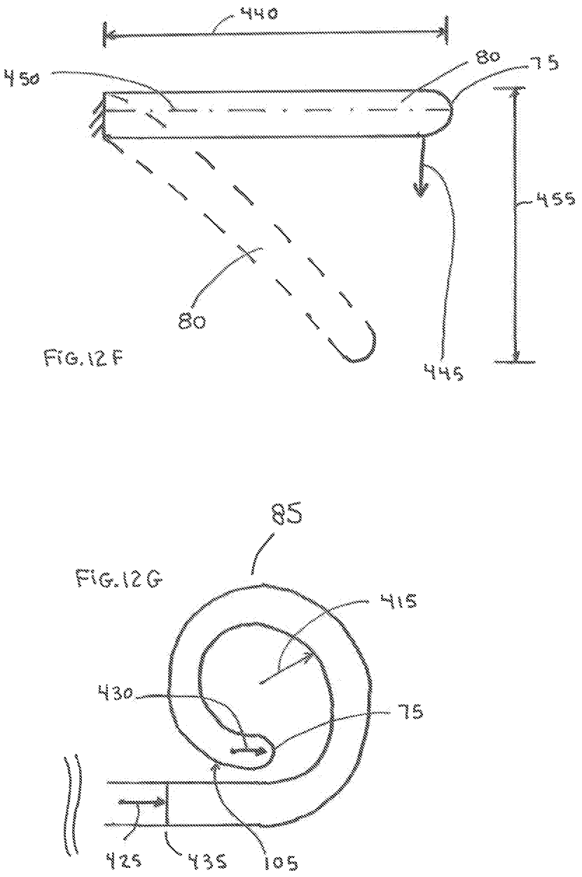

FIGS. 12A-12F show embodiments for the lead body 70 of the present invention in a lead equilibrium configuration with a lead equilibrium loop 410, i.e., a lead loop without a stylet 230 contained within the lead central lumen 235, and having a lead open loop 90. The lead body 70 has a linearly shaped lead proximal region 272 and a lead curled shaft 80 in the lead distal region 85. The lead curled shaft 80 forms a lead open loop 90 with a lead loop equilibrium radius of curvature 415 of preferably 1 cm and in the range of 0.5-2.0 cm for the lead equilibrium loop 410, i.e., the lead loop equilibrium configuration, such that a stylet 230 is not contained within the lead distal region 85. The lead equilibrium loop angle 420 for the lead equilibrium loop 410, i.e., without a stylet 230, describes the amount of curvature or curl found in the lead curled shaft 80 due to its formed equilibrium shape as shown in FIG. 12B. The lead loop angle 420 for the lead equilibrium loop 410 or other lead loop is the amount of curvature or curl as identified by the lead proximal region direction 425 relative to the lead distal end direction 430. The lead loop angle 420 as shown in FIG. 12A is 180 degrees since the loop distal end direction is 180 degrees opposed from the lead proximal region direction 425 at the lead body junction 435 between the lead proximal region 272 and lead distal region 85. The range for the lead equilibrium loop angle 420 is zero to 360 degrees; a smaller lead equilibrium loop angle 420 provides a greater advantage for removing the lead with an open loop 90 from the heart chamber 508 without entanglement with or tearing of the cordae tendineae 236. The lead loop equilibrium angle of 180 degrees, and ranging from 150-240 degrees, provides an advantageous balance between an atraumatic contact with the endocardial surface 140 during pacing and ease of removing the lead curled shaft 80 from the heart chamber 508 without tearing the cordae tendineae 236. Various lead equilibrium loop angles 420 are anticipated and depicted in FIGS. 12C-12E; FIG. 12C shows a lead distal region 85 with a lead equilibrium loop angle 420 of 270 degrees; FIG. 12D shows a lead equilibrium loop angle 420 of 90 degrees; FIG. 12E shows a lead equilibrium loop angle 420 of zero degrees; variation of the lead equilibrium loop angle 420 are understood to be included in the present invention. It is noted in FIG. 12G that the lead equilibrium loop angle 420 can be 360 degrees as shown in FIG. 12G, where the lead distal tip 250 forms an overlap portion 105 within the lead distal region 85 as described in earlier embodiments of the present invention.

The lead proximal region 272 is stiffer than the lead distal region 85; the lead proximal region 272 is able to provide the necessary push characteristics to allow the lead to be advanced within the vasculature and into the chamber of the heart 35. The lead distal region 85 has a lead bending modulus as defined by FIG. 12F of 0.6 Newtons (range 0.1-5.0 Newtons) and provides the distal region 85 with a soft and floppy characteristic that allows ease of bending. The lead bending modulus preferably ranges from 0.1-1.0 Newtons due to the more advantageous lead removal characteristics for a lead bending modulus that ranges from 0.1-1.0 Newtons. The low lead bending modulus of 0.6 Newtons (range 0.1-1.0 Newtons) allows the lead curled shaft 80 to bend and conform to the surface of the heart chamber 508 without causing tissue ischemia or necrosis at the contact of the curled shaft 80 with the endocardial surface 140. The soft, floppy lead distal region 85 allows the lead to be removed from the heart chamber 508 without entanglement with the cordae tendineae 236 and without tearing the cordae tendineae 236. As shown in FIG. 12F a lead shaft length 440 of the lead curled shaft 80 requires a lead applied force 445 acting perpendicular to the shaft central axis 450 at the lead distal end 75 to generate a lead displacement 455. The ratio of lead displacement 455 per lead shaft length 440 is equal to the lead bending strain of the lead curled shaft 80. The lead bending modulus is equal to: (lead applied force 445)/lead bending strain. Thus for the lead curled shaft 80 the amount of lead applied force 445 to cause a lead curled shaft 80 with a lead bending modulus of 0.6 Newtons to bend to a lead displacement 455 of 1 cm over a 1 cm lead shaft length 440 is 0.6 Newtons. As shown in FIG. 12F, the lead shaft length is shown to be a linear configuration for illustrational purposes. It is understood that similar principles for bending modulus determination apply to a lead curled shaft 80 with a curved or curled shape that is then further bent to an alternate radius of curvature.

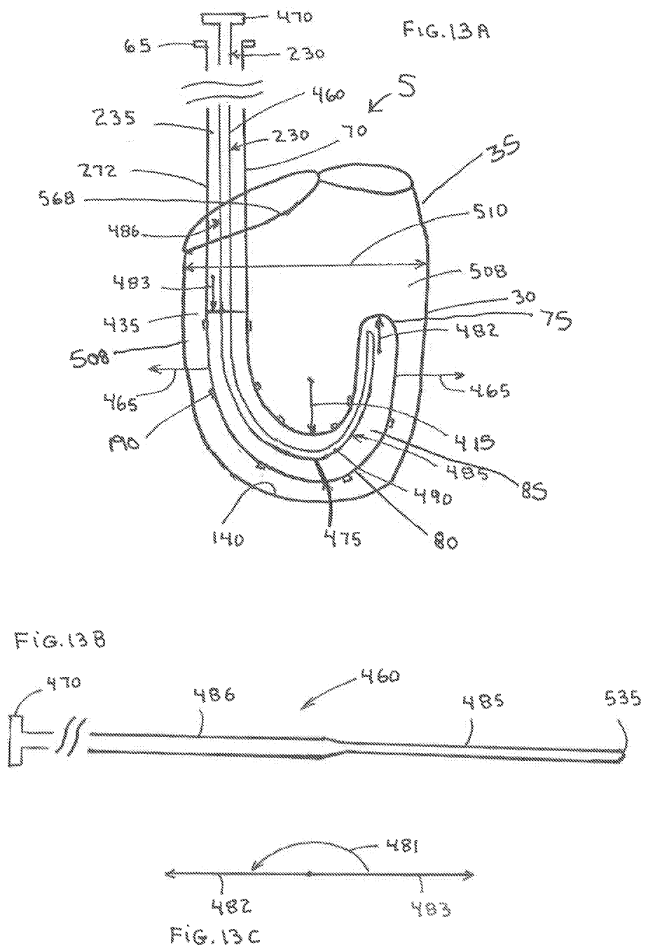

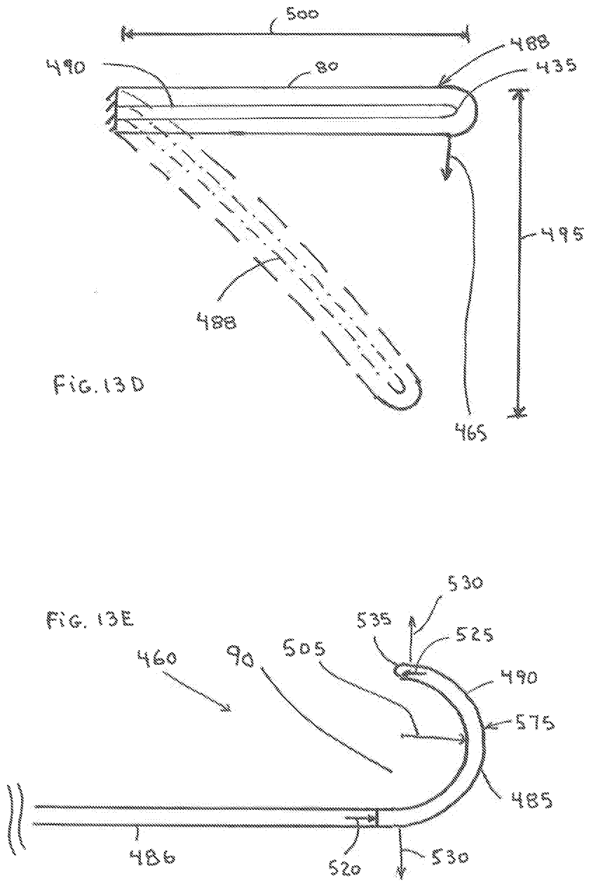

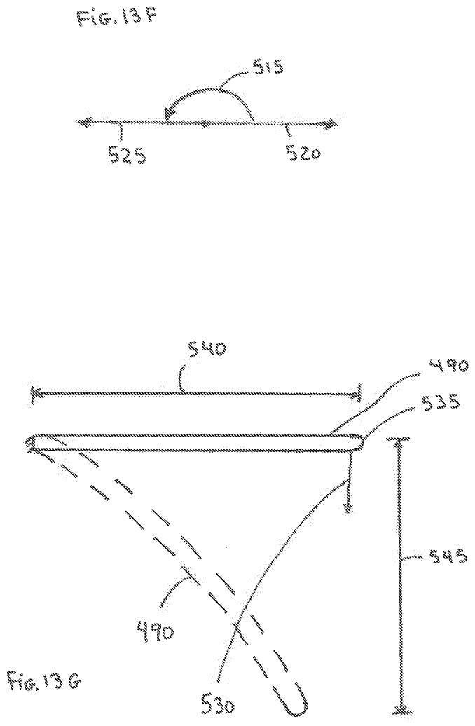

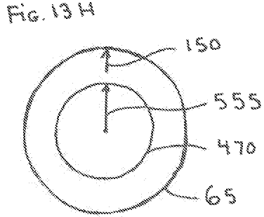

FIGS. 13A-13G show embodiments of a pacing lead 5 with a lead open loop 90 located within an RV 30 chamber 508 of the heart; a stylet 230 is located in the lead central lumen 235. The stylet 230 is a pacing stylet 460 that can be maintained within the lead central lumen 235 during pacing. FIG. 13A shows a lead distal region 85 located within the right ventricle 30 of the heart 35, for example, and making contact with the endocardial surface 140 of the RV. A straight stylet such as that shown in FIG. 13B can be placed within the lead central lumen 235 (of the lead of FIG. 12A, for example) to cause an outward lead-stylet applied force 465 onto the endocardial surface 140 to provide definite contact of the cathode sites 170 of the cathode electrode 320 with the endocardial surface 140 obtain and maintain capture of the electrical signal.

The stylet 230 has a stylet manifold 470 located at its proximal end to assist in placement depth of the stylet 230 within the lead central lumen 235 and provide rotation of the stylet 230 for rotational alignment of the stylet 230 relative to the lead body 70. The lead-stylet loop 475 has a lead-stylet loop radius of curvature 280 (with the stylet 230 inserted into the lead central lumen 235 and extending to the lead distal end 75) that is 1.5 cm (range 1.0-3.0 cm). The lead-stylet loop angle 481 for the lead-stylet loop 475 as shown in FIGS. 13A and 13C is 180 degrees to provide atraumatic contact with two walls of the endocardial surface 140 and provide an outward lead-stylet applied force 465 in opposite directions against the two opposing walls of the heart chamber 508. The lead-stylet loop angle 481 extends from the lead-stylet distal end direction 482 to the lead-stylet proximal region direction 483 and can range from 150 degrees to more than 360 degrees with the stylet 230 inserted. The lead-stylet loop radius of curvature 280 should be at least 1 cm in order to provide definite contact with the endocardial surfaces 140 on two sides of the heart chamber 508; the lead-stylet loop radius of curvature 280 can be larger, for example, with a lead-stylet loop radius of curvature 280 of up to 3.0 cm.

A straight pacing stylet 460 with a stiffer stylet proximal region that the stylet distal region 485 can provide a larger outward lead-stylet applied force of the lead curled shaft 80 onto the endocardial surface 140 of the heart 35 than a softer stylet distal region 485. The stiffer stylet distal region 485 can be obtained by a larger diameter for the stylet distal region 485 or by altering the temper of a metallic stylet or by altering the material properties of the stylet distal region 485. The outward lead-stylet applied force 465 of the combined lead curved shaft and the stylet curved shaft onto the endocardial surface 140 of the heart 35 is 0.6 Newtons (range 0.1-5 Newtons); preferably, the outward lead-stylet applied force 465 against the endocardium is 0.1-1.0 Newtons; a larger outward force against the endocardial surface 140 providers better contact of the lead distal region 80 with the endocardial surface 140 but can cause unwanted tissue ischemia and necrosis.

The outward lead-stylet applied force 465 provided by the combined material elasticity of the lead curled shaft 80 and the stylet curled shaft 490 (i.e., the lead-stylet curled shaft 488) is determined by the combined lead-stylet bending modulus of the lead curled shaft 80 and the stylet curled shaft 490 as shown in FIG. 13D. The ratio of lead-stylet displacement 495 per lead-stylet shaft length 500 is equal to the lead-stylet bending strain of the lead-stylet curled shaft 488. The lead-stylet bending modulus is equal to: (lead-stylet applied force 465)/lead-stylet bending strain. Thus for the lead-stylet curled shaft 488 the amount of lead-stylet applied force 465 to cause a lead-stylet curled shaft 488 with a lead-stylet bending modulus of 0.6 Newtons to bend to a lead-stylet displacement 495 of 1.5 cm over a 1.5 cm shaft length is 0.6 Newtons. The bending modulus for the stylet distal region 485 is 0.6 Newtons (range 0.1-5.0 Newtons, and preferably 0.1-1.0 Newtons) such that the lead-stylet outward applied force against the endocardium is controlled to 0.6 Newtons (range 0.1-1.0 Newtons). As shown in FIG. 13D, the lead-stylet shaft length 500 is shown to be a linear configuration for illustrational purposes. It is understood that similar principles for bending modulus determination apply to a lead-stylet curled shaft 488 with a curved or curled shape that is then further bent to an alternate radius of curvature.

The pacing stylet 460 can have other configurations other that those shown in FIG. 13B. For example, as shown in FIG. 13E, the pacing stylet 460 can have a stylet radius of curvature 505 of 2.0 cm and can be placed into a lead open loop 90 with a lead loop equilibrium radius of curvature 415 of 1 cm such as shown in FIG. 12A and form a lead-stylet loop 475 with a lead-stylet loop radius of curvature 280 or 1.5 cm, for example, as shown in FIG. 13A. A lead-stylet radius of curvature of preferably 1.5 cm with a range of 1-3 cm will provide suitable contact of the pacing lead 5 with the endocardial surface 140 of the heart chamber 508 and provide electrical signal capture. The stylet radius of curvature 505 can range from 1 cm (for a lead loop equilibrium radius of curvature 415 that is larger than 3 cm, for example) to infinity or a straight stylet, for example, for use in a lead loop equilibrium radius of curvature 415 that is smaller than the heart chamber width 510. The average heart chamber width 510 at a location of contact with the pacing lead body 70 is typical 3 cm with a range of 1-5 cm. The pacing stylet 460 can have a stylet loop angle 515 as described by FIG. 13F that extends from a stylet proximal region direction 520 to a stylet distal end direction 525 of preferably 180 degrees with a range of zero to 360 degrees.

The stylet has a stylet bending modulus that is determined by a stylet applied force 530 causing the stylet distal end 535 to bend over a stylet shaft length 540 of the stylet shaft as shown in FIG. 13G. The ratio of stylet displacement 545 per stylet shaft length 540 is equal to the stylet bending strain of the stylet curled shaft 490. The stylet bending modulus is equal to: (stylet applied force 530)/stylet bending strain. Thus for the stylet curled shaft 490 the amount of stylet applied force 530 to cause a stylet curled shaft 490 with a stylet bending modulus of 0.6 Newtons to bend to a stylet displacement 545 of 1 cm over a 1 cm stylet shaft length 540 is 0.6 Newtons. As shown in FIG. 13F, the stylet shaft length is shown to be a linear configuration for illustrational purposes. It is understood that similar principles for bending modulus determination apply to a stylet curled shaft 490 with a curved or curled shape that is then further bent to an alternate radius of curvature.

The stylet of the present invention can have a stylet bending modulus in the stylet distal region 485 that ranges from 0.1-5 Newtons and preferably ranges from 0.1-1.0 Newtons to more closely equal and balance the bending modulus of the lead distal region 85 and provide a suitable outward lead-stylet applied force 465 that does not generate trauma to the endocardial surface 140. The outward lead-stylet applied force 465 will also provide a more linear relationship with respect to lead-stylet displacement 495 if the lead bending modulus and lead loop equilibrium radius of curvature 415 is similar in magnitude to the stylet bending modulus and stylet radius of curvature 505. Thus, the stylet radius of curvature 525, the stylet bending modulus, and the stylet loop angle 515 are combined with the lead loop equilibrium radius of curvature 415, the lead bending modulus, and the lead equilibrium loop angle 420 to determine the lead-stylet loop radius of curvature 480, the lead-stylet loop angle 481, and the outward lead-stylet applied force 465 onto the endocardial surface 140 of the heart 35.

The outward stylet applied force 530 can act in the same outward direction as an outward lead applied force 445, acting against the endocardial surface 140, and hence the two forces are addictive. If the lead curled shaft 80 is of a smaller lead loop radius of curvature than the stylet loop radius of curvature, then the stylet applied force 530 can be acting to enlarge the lead loop radius of curvature and the lead applied force 445 is acting in a direction opposed to the stylet curled shaft 490. The outward forces of the lead applied force 445 and the stylet applied force 530 are expected in the present invention to provide a combined outward lead-stylet applied force 465 onto the endocardial surface 140 of 0.6 Newtons (range 0.1-5.0 Newtons, and preferred range of 0.1-1.0 Newtons) to ensure that tissue ischemia and necrosis of the myocardial tissues 145 are not generated.

For the embodiment wherein the lead curled shaft 80 has a lead loop equilibrium radius of curvature 415 of 1 cm and a stylet has a stylet radius of curvature 505 of 2 cm as described in FIG. 13E, the lead-stylet loop radius of curvature 280 could be 1.5 cm provided that the stylet curled shaft 490 aligned in the same plane and with the same coil direction as the lead curled shaft 80. Such alignment tends to occur naturally as the operator inserts the stylet into the lead central lumen 235. However, if the operator is intending to place the stylet into the lead central lumen 235 such that the lead curled shaft 80 is directly opposed to the stylet curled shaft 490 (i.e., the direction of the stylet loop forms a spiral in a direction that is opposite to the lead loop direction) then an indicator can be located on the stylet manifold 470 and the lead manifold 65 to assist the operator in obtaining the alignment direction that is desired to obtain contact of the lead distal region 80 with the endocardial surface 140 without causing tissue ischemia or necrosis. As shown in FIG. 13H a lead indicator 150 located on the lead manifold 65 can be oriented relative to a stylet indicator 555 located on the stylet manifold 470 to place the orientation of the lead curled shaft 80 in alignment with the stylet curled shaft 490. A locking means such as a screw-type mechanism can be incorporated into the lead indicator 150 and stylet indicator 555 to lock a specific orientation.

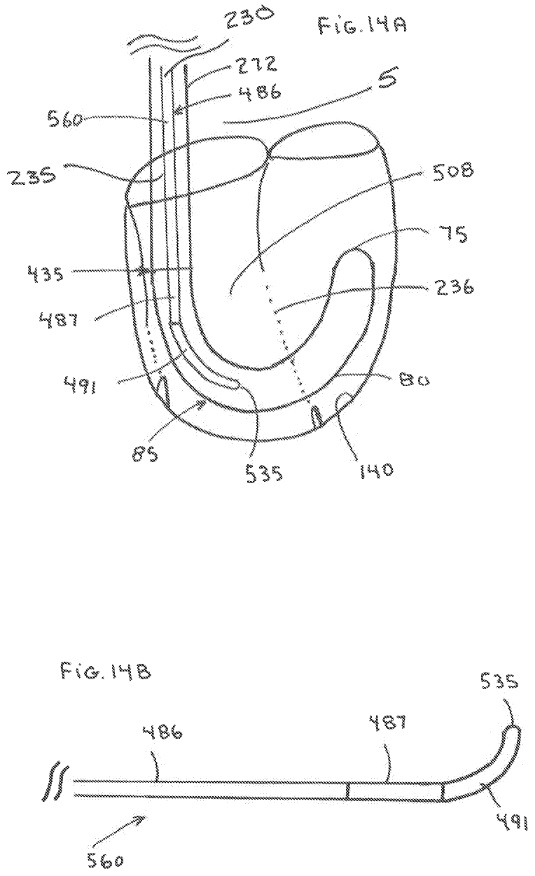

Removal of the temporary pacing lead 5 from the chamber of the heart 35 is accomplished by inserting a stylet 230 that can be a removal stylet 560 such as that shown in FIGS. 14A and 14B into the lead central lumen 235 as shown in FIG. 14A. The stylet has a linearly configured stylet proximal region 486 and a linear stylet central region 487 that extends within a portion of the lead distal region 85. The removal stylet 560 has a curled stylet distal tip 535 that is of a lower bending modulus than the stylet proximal region 486 or stylet central region 487; the stylet distal end 535 can be advanced to a position short of approximation with the lead distal end 75. The stylet serves to straighten a portion of the lead distal region 85 located in the chamber of the heart 35; the stylet distal tip serves as a transition region to allow the lead to be retracted under tension over the stylet 230 until the stylet distal end 535 contacts the lead distal end 75 and further retracted out of the heart chamber 508 along with the stylet 230 such that the lead curled shaft 80 does not entangle with cordae tendineae 236 of the heart chamber 508 as the lead is being retracted under tension.

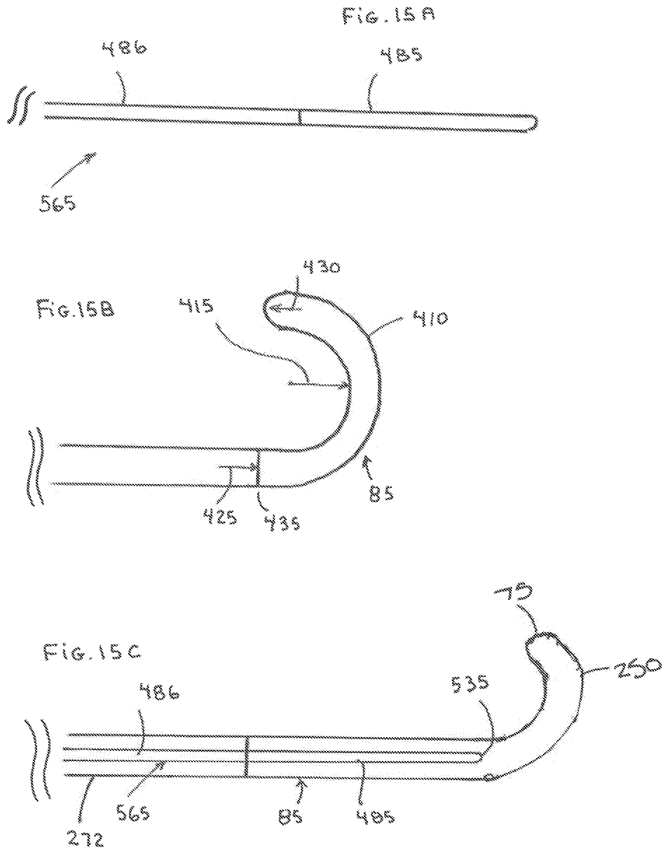

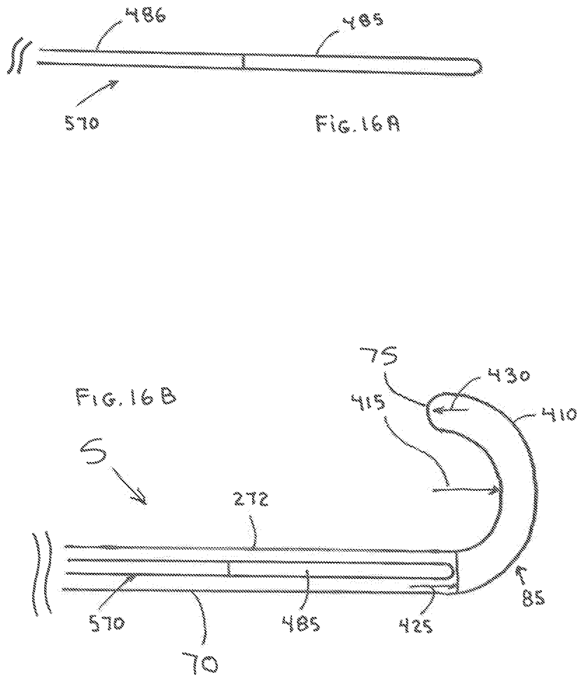

Introduction of the temporary pacing lead 5 into the vasculature requires that much of the lead body 70 is generally straight except for a curved lead distal tip 250 that can help to negotiate turns within the vasculature and prolapse safely across the TCV. To accomplish the traversal within the vasculature, a generally straight vascular stylet 565 as shown in FIG. 15A can be inserted, for example, into a lead body 70 with an equilibrium configuration as shown in FIG. 15B to provide a combined lead-stylet configuration as shown in FIG. 15C. The lead loop equilibrium radius of curvature 415 can be, for example, 1 cm. The stylet can be stopped proximal to the lead distal tip 75 such that the curled shape of the lead curled shaft provides a small curvature that enables negotiation of vascular turns.

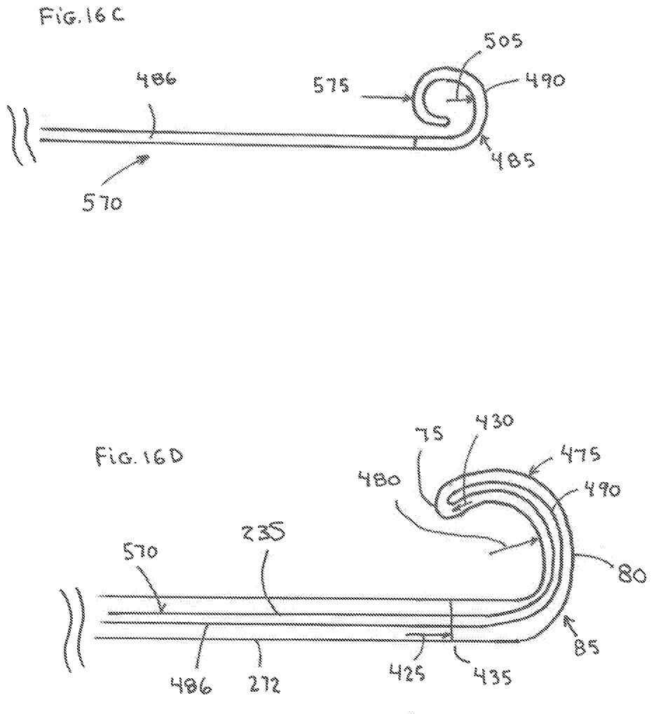

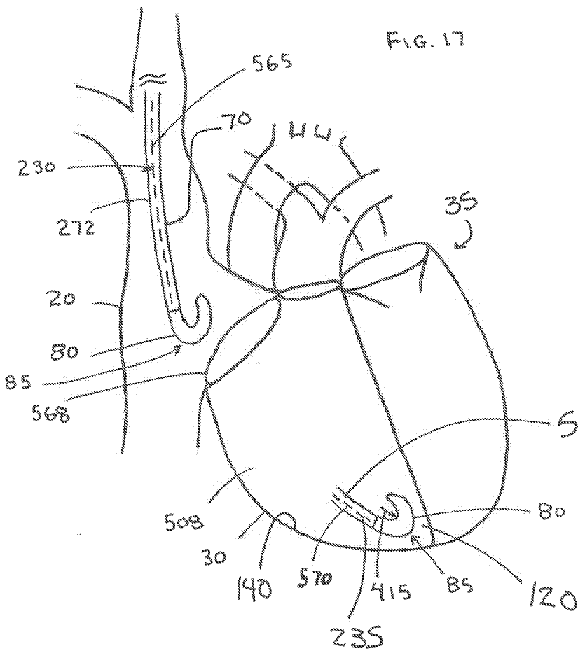

Advancement of the temporary pacing lead 5 into the chamber of the heart 35 requires that the configuration of the curled shaft 80 be rounded and atraumatic to the endocardial surface 140. A proximal secondary bend in the catheter or stylet can give directionality to the lead directing it toward the tricuspid valve annulus and thus entry into the RV. Also, the lead curled shaft 80 must be suitable to traversing the vasculature with a curled shaft 80 suitable to traverse the annulus 568 of the heart 35 and enter the heart chamber 508. The lead loop equilibrium radius of curvature 415 of 1 cm allows the lead curled shaft 80 to form the lead loop within the RA. Withdrawal of the vascular stylet 565 (while maintaining a fixed position for the lead body 70) which can then also serve as a ventricular placement stylet 570 as shown in FIGS. 16A and 16B allows the lead distal region 85 to form a lead equilibrium loop 410 that is suitable (in both a small radius of curvature and a rounded shape) for entering into the heart chamber 508. The lead curled shaft 80 must fit through a 2 cm diameter annulus 568 leading into the ventricular chamber and must have an atraumatic curled configuration that cannot produce trauma to TCV 25 or the endocardial surface 140 of the heart chamber 508. Alternately, to obtain a lead-stylet loop radius of curvature 280 that is smaller than the lead loop equilibrium radius of curvature 415, a shaped ventricular placement stylet 570 having a stylet loop 575 in the stylet curled shaft 490 with a stylet radius of curvature 505 of 0.5 cm, for example, can be introduced into the lead central lumen 235 to cause the lead-stylet loop radius of curvature 480 to be 0.75 cm, for example, as shown in FIGS. 16C and 16D.

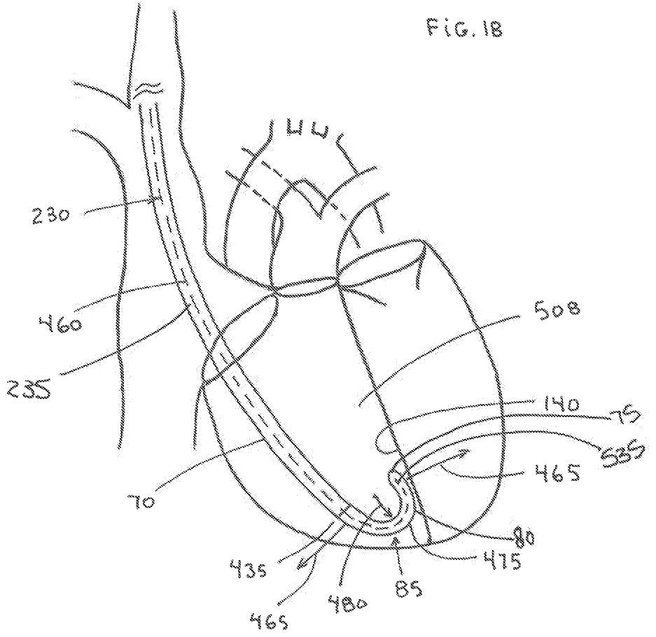

The method of use for the temporary pacing lead 5 of the present invention is shown in FIGS. 17-19. In FIG. 17 the lead distal region 85 has been delivered to the RA 20 and the vascular stylet 565 has been withdrawn allowing the lead curled shaft 80 to form an atraumatic shape within the RA as shown in the RA 20 portion of FIG. 17. A ventricular placement stylet 570 may be introduced into the lead central lumen 235, if desired, to form a different lead-stylet curled loop. The pacing lead 5 is then advanced to the apex 120 of the right ventricle, RV 30 also shown in the RV 30 portion of FIG. 17. The lead curled shaft 80 of the lead distal region 85 may obtain a lead loop equilibrium radius of curvature 415 if a stylet is not introduced into the lead distal region 85. If the loop equilibrium radius of curvature can provide capture of an electrical signal to the myocardium, then temporary pacing is initiated. If additional outward force or additional outward displacement is needed by the lead curled shaft 80 to make contact with the heart chamber surface, then a pacing stylet 460 is introduced into the lead body central lumen 235 as shown in FIG. 18. The pacing stylet 460 can increase (or decrease; a decrease occurring if the stylet radius of curvature 505 is smaller than the lead loop equilibrium radius of curvature 415) the lead-stylet loop radius of curvature 280 and increase (or decrease; a decrease occurs if the stylet radius of curvature 505 is smaller than the lead-stylet loop radius of curvature 480) the outward force placed onto the endocardial surface 140. Upon completion of temporary pacing, a removal stylet 560 can be placed into the lead central lumen 235 such that a generally linear portion of the stylet extends into the lead distal region 85 to help straighten the lead curled shaft 80 and assist with ease of lead removal. The lead body 70 can be withdrawn proximally under tension while maintaining position of the removal stylet 560 within the lead central lumen 235. The lead curled region distal end 75 is withdrawn toward the stylet distal end 535 such that the lead curled region does not entangle or tear the cordae tendineae 236 as shown in FIG. 19; then, the pacing lead body 70 and stylet 230 can be withdrawn together out of the heart chamber 508. The soft, floppy lead distal region 85 allows the pacing lead 5 to be removed from the heart chamber 508 without entangling the cordae tendineae 236.

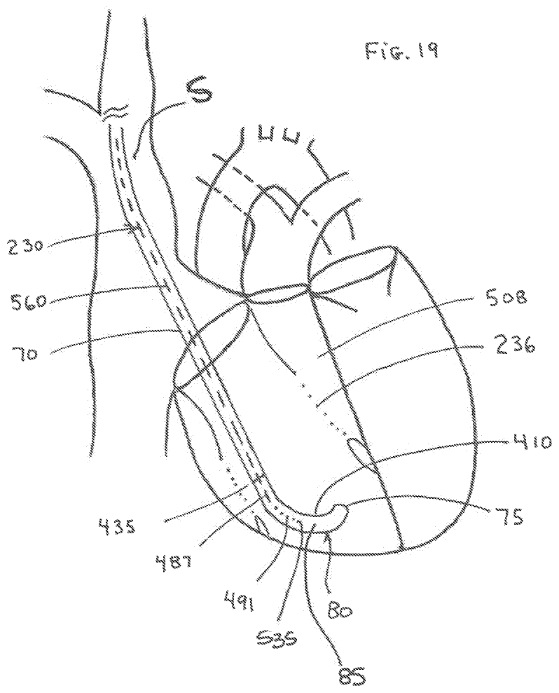

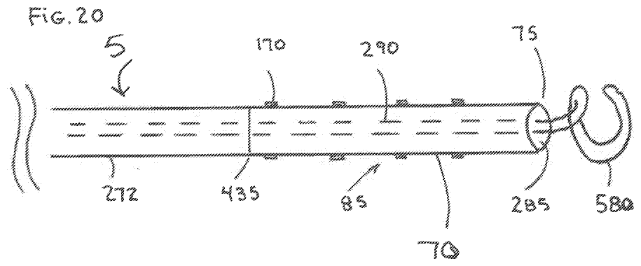

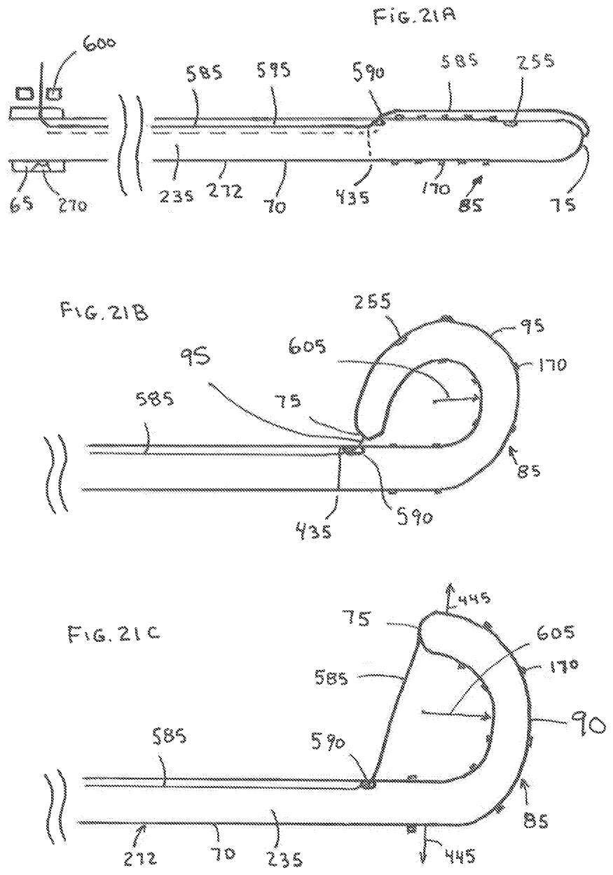

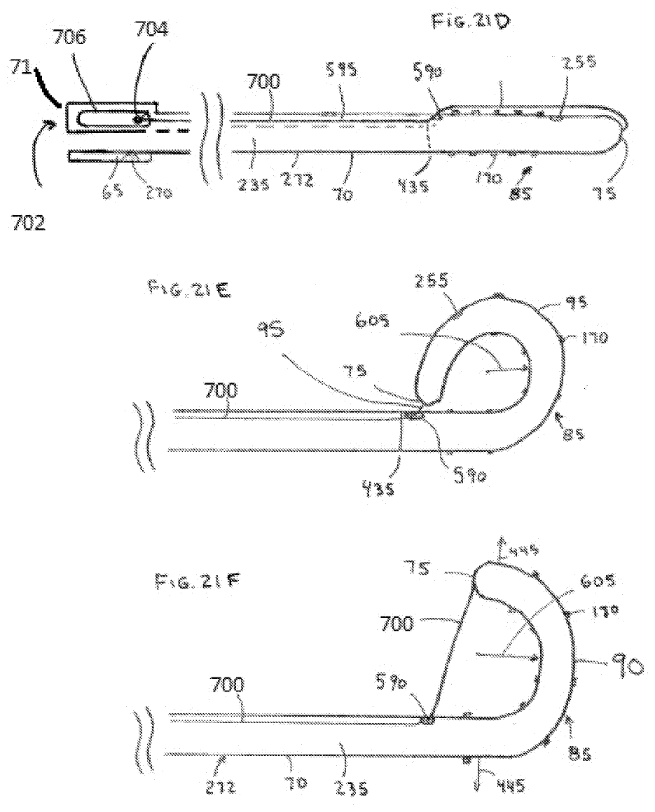

The temporary pacing lead 5 having an open loop 90 can have an open distal end 285 as shown in FIG. 20; the lead structure for this embodiment has been described earlier in FIGS. 8A-8C and 9A-9B and in other embodiments. The stylet that has been described in earlier embodiments to alter the lead loop radius of curvature and alter the outward forces provided by the lead distal region 85 against the myocardial surface can be a guidewire 290. The stylet 230 can be a shaped guidewire 290 that provides atraumatic passage of the lead body 70 over the guidewire 290 through the vasculature, into the heart chamber 508, and during removal of the pacing lead 5 from the chamber of the heart 35. The guidewire 290 can also have a guidewire coiled distal tip 580 or pig-tail, the guidewire coiled tip can be placed adjacent and distal to the lead distal end 75 to provide an atraumatic lead-wire configuration to the lead distal end 75 that allows the lead and guidewire 290 to be advanced together into the chamber of the heart 35 without a need for fluoroscopic guidance.

### Control Fiber 585

A further embodiment for the pacing lead 5 of the present invention having multiple electrodes 170, distal pressure measuring capability, and a lead closed loop 95 is shown in FIGS. 21A-21C. FIG. 21A shows the lead distal region 85 of the lead body 70; a control fiber 585 is attached to the lead distal end 75 and traverses external to the lead distal region 85. The control fiber 585 enters a control opening 718 590 at the lead body junction 435 of the lead proximal region 272 and lead distal region 85. The control fiber 585 extends through a control lumen 595 within the lead proximal region 272 to a lead manifold 65 located at the proximal end of the lead body 70. A holding-tensioning member 600 is attached to the lead manifold 65. The holding-tensioning member 600 is able to take up length of the control fiber 585 and provide tension to the control fiber 585. As shown in FIG. 21A, the lead body 70 is in a linear configuration to traverse the introducer sheath 10. A stylet 230 can be introduced into the lead body central lumen 235 as described in earlier embodiments. The control fiber 585 provides a tension from the lead distal end 75 to the control opening 718 590 that provides a lead loop-controlled radius of curvature 605 to the lead distal region 85 of the lead body 70 as illustrated in FIG. 21B.

Once the lead distal end 75 has traversed through the vasculature and reached the right atrium 20 or the annulus 568 leading to the heart chamber 508, the control fiber 585 can be activated by applying tension via the holding-tensioning member 600. Application of tension causes the lead distal region 85 to form a closed loop 95 as shown in FIG. 21B. The closed loop 95 has a lead loop-controlled radius of curvature 605 that allows entry of the closed loop 95 into the chamber of the heart 35 in an atraumatic manner.

Once the lead distal region 85 has been advanced into the chamber of the heart 35, the control fiber 585 can be released to allow the lead distal region 85 to form a curled shaft 80 having loop 90 which remains closed by virtue of the tensioning fiber attachment at the distal lead tip 75 and a fiber controlled opening 590 as shown in FIG. 21C. The outward lead applied force 445 provided by the lead distal region 85 due to the bending energy stored in the lead distal body 70 causes contact between the electrode sites 170 and the endocardial surface 140. The outward lead applied force 445 can be adjusted by introducing a stylet, if necessary, as described in earlier embodiments to provide an outward force of preferably 0.6 Newtons with a range of 0.1-5.0 Newtons. The outward applied force is preferred to have an upper range limit of 1.0 Newtons to ensure that the heart chamber 508 tissue does not become ischemic. Alternatively, the control fiber 585 can be released an additional amount or can be retracted under tension to alter the outward lead applied force 445 inherent in the stored bending energy of the lead distal region 85 onto the endocardial surface 140.

Removal of the pacing lead 5 is accomplished by applying tension to the control fiber 585 via the holding-tensioning member 600 to place the lead distal region 85 into a closed loop 95 as shown in FIG. 21B. The pacing lead 5 can be pulled back under tension into the RA 20 where the control fiber 585 can be released to allow the lead distal region 85 to form a linear configuration as shown in FIG. 21A. Alternately, the control fiber 585 can be released of all tension within the heart chamber 508 allowing the lead body 70 to assume a more linear shape similar to that of FIG. 21A prior to removing the lead body 70 from the heart chamber 508. Since the lead distal tip 250 is attached to the lead body 70 via the control fiber 585 at the control fiber opening 590, entanglement of the lead distal region 85 with cordae tendineae 236 is obviated.

Still another embodiment for the pacing lead 5 of the present invention having multiple electrodes 170, distal pressure measuring capability, and a lead closed loop 95 is shown in FIGS. 21D-21F. FIG. 21D shows the lead distal region 85 of the lead body 70; a push-pull element 700 is attached to the lead distal end 75 and traverses external to the lead distal region 85. The push-pull element 700 enters a control opening 718 590 at the lead body junction 435 of the lead proximal region 272 and lead distal region 85. The push-pull element 700 extends through a control lumen 595 within the lead proximal region 272 to a lead manifold 65 located at the proximal end 71 of the lead body 70. A tension-compression member 702 is attached to the lead manifold 65. The tension-compression member 702 is able to take up length of the push-pull element 700 by application of tension to the push-pull element 700 to form the distal region into a curled shaft as illustrated in FIG. 21E; the tension-compression member 702 is also able to provide length to the push-pull element 700 by applying a compressive force onto the push-pull element 700 that forces the push-pull element 700 distally toward the lead distal end 85 and form the distal end 85 into a straightened configuration suitable for lead passage through the vasculature or removal of the lead from the heart chamber without trauma to the cordae tendineae.

The push-pull element 700 is able to push the curled shaft 80 outwards against an endocardial wall that opposes the endocardial surface making contact with the lead body in a heart chamber to generate a lead applied force 445 that ensures that one or more electrodes 202 make full contact with the endocardial surface 140 of the heart 35 and signal capture is attained and lead thresholds are optimized. The outward applied force is controlled by the compressive forces generated within the push-pull element 700 by the operator and transferred to the curled shaft 80 and further transferred to the endocardial surface 140 of the heart 35.

The tension-compression member 702 comprises a movable member 704 that forms an attachment with the push-pull element 700. Movement of the movable member 704 by the operator causes the push-pull element 700 to move proximally under tension or distally under compression. The tension-compression member 702 further comprises a constraining member 706 that provides a housing and appropriate constraining forces onto the movable member 704 to hold the movable member 704 at a desired location. The movable member 704 can be a slide, a circular spool, or other member that is able to move either linearly or rotationally and affect the position of the push-pull element 700. The constraining member 706 can be a slotted member that constrains or holds the movable member 704 via frictional forces, for example, a ratchet that constrains movement of a rotational movable member 704, for example, or other constraining member 706 such as a locking mechanism capable of holding the movable member 704 via frictional forces or via other holding method used to hold a compression force or tension force placed in the push-pull element 700.

The push-pull element 700 can be a metal or polymeric ribbon or wire that is able to provide both tension to the lead distal end 75 or provide a compressive force that pushes the lead distal end 75 away from the manifold 160; movement of the lead distal end 75 via compression of the push-pull element 700 can assist in placing the lead distal region into contact with the endocardial surface 140 or can place the lead distal region into a more linear configuration suitable for removal of the temporary lead from the body. The push-pull element 700 could be formed from a stainless steel ribbon, Nitinol ribbon, or other metal or polymeric ribbon or wire that is able to both pull the lead distal end 75 proximally or push the lead distal end 75 distally to a more straightened configuration.

As shown in FIG. 21D, the lead body 70 is in a linear configuration to traverse the introducer sheath 10. A stylet 230 can be introduced into the lead body central lumen 235 as described in earlier embodiments. The push-pull element 700 provides a tension from the lead distal end 75 to the control opening 718 590 that provides a lead loop-controlled radius of curvature 605 to the lead distal region 85 of the lead body 70 as illustrated in FIG. 21E.

Once the lead distal end 75 has traversed through the vasculature and reached the right atrium 20 or the annulus 568 leading to the heart chamber 508, the push-pull element 700 can be activated by applying tension via the tension-compression member 702. Application of tension causes the lead distal region 85 to form a closed loop 95 as shown in FIG. 21E. The closed loop 95 has a lead loop-controlled radius of curvature 605 that allows entry of the closed loop 95 into the chamber of the heart 35 in an atraumatic manner.

Once the lead distal region 85 has been advanced into the chamber of the heart 35, the push-pull element 700 can be placed under compression to cause the lead distal region 85 to form a curled shaft 80 having loop 90 which is still considered to be a closed loop since the push-pull element 700 is attachment at the distal lead tip 75 and a control opening 718 590 forming a complete loop as shown in FIG. 21F. The outward lead applied force 445 provided by the lead distal region 85 is due to the compressive forces of the push-pull element 700 being applied to the lead distal body 70 as well as due to the curled shaft applied force 225 described earlier generated by the elastic modulus of the curled shaft material. The outward lead applied force 445 causes contact between the electrode sites 170 and the endocardial surface 140. In one embodiment the curled shaft 80 can be formed with a linear equilibrium configuration and the curled shaft applied force 225 can be combined with the compressive forces generated by the push-pull element 700 to generate the outward lead applied force 445 as shown in FIG. 21F that is greater than can be attained with the use of a control fiber. In another embodiment, the curled shaft 80 can be formed with a curved equilibrium configuration and the push-pull element 700 can apply a compressive force to straighten the curled shaft 80 during passage through the vasculature, for example.

The outward lead applied force 445 can be adjusted by moving the movable member 704 to push the push-pull element 700 distally and moving the lead distal end 75 into contact with the heart chamber tissue to provide an outward force of preferably 0.6 Newtons with a range of 0.1-5.0 Newtons. The outward applied force is preferred to have an upper range limit of 1.0 Newtons to ensure that the heart chamber 508 tissue does not become ischemic. Alternatively, the push-pull element 700 can be placed under tension an additional amount by movement of the movable member 704 to alter the outward lead applied force 445 that is applied onto the endocardial surface 140.

Removal of the pacing lead 5 is accomplished by applying compression to the push-pull element 700 causing the lead body 70 to assume a more linear shape similar to that of FIG. 21D prior to removing the lead body 70 from the heart chamber 508. Since the lead distal tip 250 is attached to the lead body 70 via the push-pull element 700 at the control opening 718 590, entanglement of the lead distal region 85 with cordae tendineae 236 is obviated.

Providing a push-pull element 700 that transfers both tension and compression to the lead distal end 75 rather than only a control fiber that applies only tension to the lead distal end 75 alters the operation and capability of the temporary lead in several unique ways. With a control fiber that can only apply tension the outward applied force 225 is controlled by the elastic modulus or bending modulus of the lead distal region 85. The elastic modulus and outward applied force is dependent upon the choice of polymer for the lead distal region 85, the durometer of the polymer, the diameter, and length of the lead distal region 85. A larger diameter distal region would generate a larger outward applied force, for example; a lower durometer polymer with a lower modulus would generate a lower outward applied force by the lead distal region 85 against an opposing endocardial surface of the heart chamber, for example. The outward applied force generated by the lead bending modulus can be less than 0.1 Newtons, for example, to provide a very atraumatic curled region; application of compression by the push-pull element 700 can generate an outwardly directed curled shaft applied force of 0.6 Newtons to provide improved contact of the electrodes 190 with the endocardial surface and reduce threshold currents needed to obtain capture of the electrical signal from the electrode 190 to the heart tissue.

With the push-pull element 700 rather than only a control fiber, the outward applied force is far less dependent upon the elastic modulus and dimensions for the lead distal region 85. The outward applied force is generated by the compressive force found in the push-pull element 700 which is controlled by the operator. The operator can push the movable member 704 of the tension-compression member 702 toward the lead distal end 75 to place the push-pull element 700 into greater compression which then generates a greater outward applied force by the lead distal end 75 to an opposing endocardial wall that is opposite the endocardial surface that is in contact with the lead body 70 of a heart chamber.

With the use of the push-pull element 700 rather than the control fiber, the curled shaft 80 of the temporary lead can be formed from a lower modulus polymer or of a polymer of lower durometer than can be used with only the control fiber and generate a greater outward applied force against the opposing wall of the heart chamber such as the septal wall, for example. The use of a lower modulus polymer or lower durometer polymer in the lead distal region 85 allows the curled shaft 80 to be more supple and more easily removed from the heart chamber in a configuration similar to that of FIG. 21D without causing trauma to the cordae tendineae. A more flexible distal region 85 allows the lead distal end 75 to be more safely advanced across the cordae tendineae and into the apex of the heart, yet still achieve a larger outward lead applied force 445 against opposing wall of the heart chamber with the push-pull element 700 rather than with the control fiber.

With the use of the push-pull element 700 rather than the control fiber the catheter distal region 85 can form a curled shaft with a larger radius of curvature that is generated by the compressive characteristics of the push-pull element 700. The push-pull element 700 can push the curled shaft outwards to make contact with an opposing wall of the heart chamber such as the septal wall, for example, that is further away from a lateral wall of the right ventricle, for example. The outward applied forces 445 provided by the curled shaft 80 for the larger heart chamber are controlled by the compressive forces provided by the push-pull element 700; such outward applied force would be less for a lead distal region 85 formed of a lower modulus material having only a control fiber which operates only under tension.

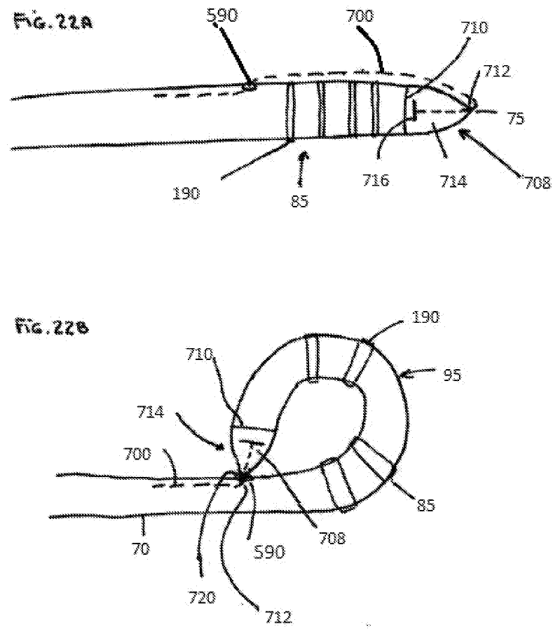

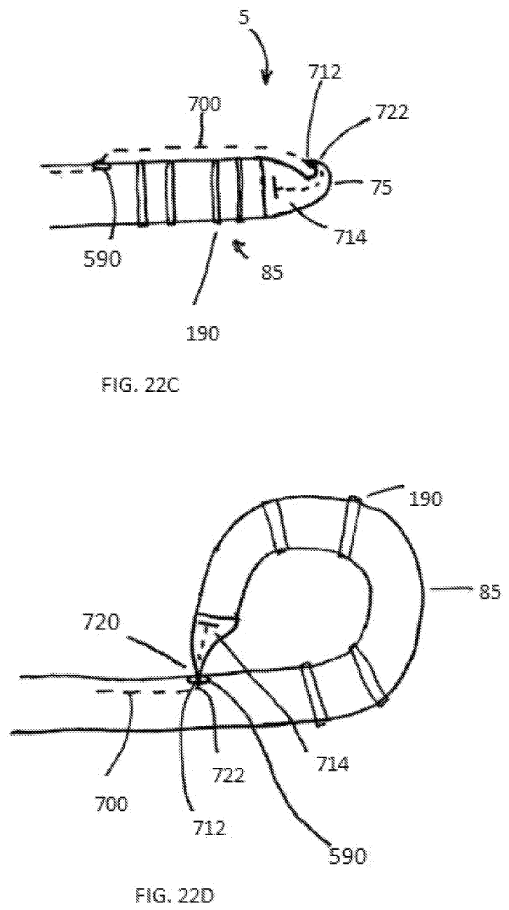

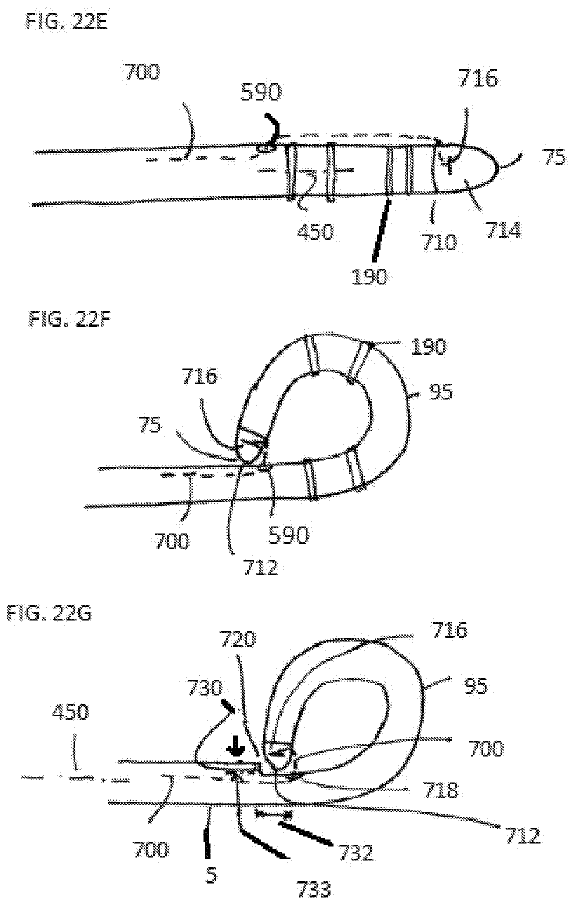

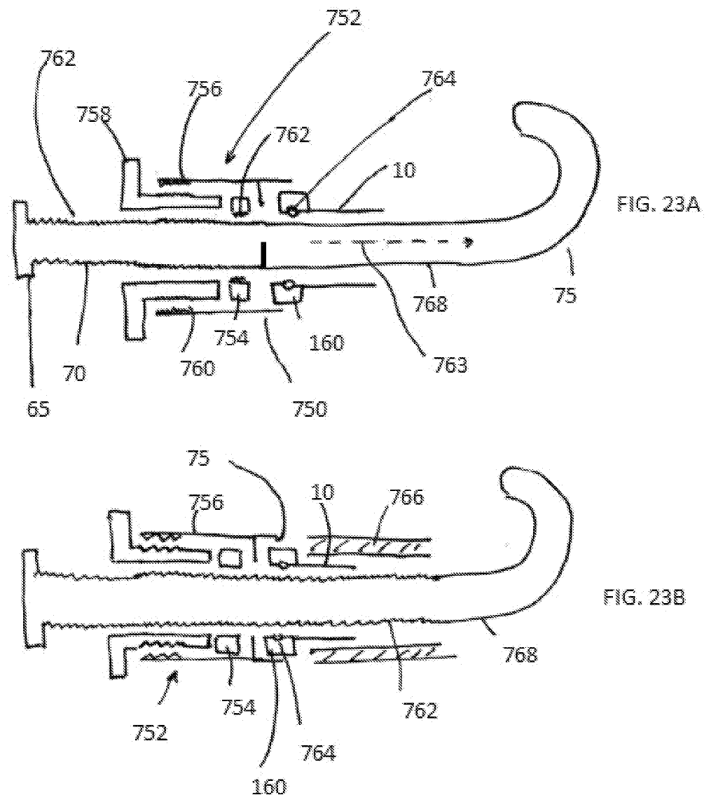

The embodiment for the pacing lead 5 of the present invention shown in FIGS. 21A-21F can have a lead distal region 85 having a variety of configurations as shown in lead distal region. As shown in FIGS. 22A and 22B a distal feature 714 in the form of a soft conical tip 708 can extend from a feature base 710 to a feature distal end 712. The conical tip 708 can be formed with a generally pointed distal end 75 which has the control fiber 585 as shown in FIGS. 21A-21C or push-pull element 700 as shown in FIGS. 21D-21F extending within and attached to the conical tip 708. The push pull element 700 (or control fiber 585) can be anchored within the distal feature 714 by attachment to a holding element 716 that is embedded within the distal feature 714. The holding element 716 can be a metallic pin or rod, for example, to which the push pull element 700 can be secured via a knot, adhesive, or other attachment method. Such a conical tip 708 can be formed by thermal processing, insert molding, or via other processing methods to form a generally conical or smooth geometric taper that extends from the feature base 710 to the feature distal end 712 which forms an apex to the conical tip 708 at the lead distal end 75. As shown in FIG. 22A the push-pull element 700 or control fiber 585, as seen in FIGS. 21A-21C, extends out of the feature distal end 712 and extends along the outside surface of the lead distal region 85 and enters the control opening 718 as described in FIGS. 21A-21C. As tension is placed into the push-pull element 700 or control fiber 585 as shown in FIG. 22B by a holding/tensioning element or constraining member 706 as described in prior embodiments, the lead distal region 85 is formed into a closed loop and the feature distal end 712 is brought into intimate contact with the control opening 718. A portion of the feature distal end 712 of the conical tip 708 in some embodiments can enter the control opening 718 thereby forming a smooth surface transition between the lead body 70 and the distal cone 714 at the tip junction 720. This smooth transition obviates any concern to the operator that a cordae tendineae or native leaflets could become entangled at the tip junction 720 between the feature distal end 712 and the control opening 590 that could occur upon withdrawal or removal of the temporary lead 5 from the chamber of the heart.

An alternate configuration for the distal feature 714 for the temporary pacing lead 5 is shown in FIGS. 22C and 22D. In this configuration a small tail 722 is formed into the feature distal end 712. The tail 722 can be formed out of a similar polymer or from a softer polymer with a lower durometer than that used to form the remainder of the distal feature 714; such polymers for the distal feature 714 or the tail 722 include polyolefin, silicone, polyurethane, polyester, Pebax, and other materials used to form catheter shafts and tips used in pacing leads and angioplasty catheters. A tail 722 that is formed from a soft resilient material will allow the tail 722 to form a generally curved shape with an rounded shape for the lead distal end for atraumatic use during the delivery of the pacing lead 5 through the vasculature and upon initial entry of the temporary lead into some chambers of the heart or other body cavity or lumen. The push-pull element 700 or control fiber 585 can extend within the interior of the distal feature 714, extend through the interior of the tail 722, and extend further along the outside of the lead distal region 85 until the push-pull element 700 or control fiber 585 extends into the control opening 718. The tail 722 can be formed with the push-pull element 700 or control fiber 585 embedded therein via thermal processing, solvent processing, insert molding, and other processing methods. Once the pacing lead 5 is ready for forming the closed loop, tension is applied to the push-pull element 700 or control fiber 585 and the tail 722 is able to straighten out and become directed in line with the push-pull element 700 or control fiber 585 and with approximate curvature with the distal region of the closed loop and can extend into contact with or extending within the control opening 718 as shown in FIG. 22D. After the closed loop is formed, the tail 722 forms a smooth transition to the lead distal region 85 at the tip junction 720 to ensure that cordae tendineae or leaflets are not snagged at the tip junction 720.

FIGS. 22E-22G show an embodiment having the push-pull element 700 or control fiber 585 attached at or near a location near the feature base 710 of the distal feature 714 rather than at the feature distal end 712. The distal feature 714 can be a rounded tip, a smooth asymmetrical tip, other smooth tip shape, or a conical tip 708. The push-pull element 700 or control fiber 585 enters the distal feature 714 at or near the feature base 710 of the distal feature 714 and is anchored via attachment to a holding element 716. Upon application of tension to the push-pull element 700 or control fiber 585, the distal region is formed into a closed loop as shown in FIGS. 22F and 22G. In FIG. 22F the feature distal end 712 or the distal feature 714 near the feature distal end 712 is brought into direct contact with the lead body 70 to make a contact of the distal feature 714 with the lead body 70 near the control opening 718. A small recess 730 can be formed into the lead body 70 as shown in FIG. 22G to have a smooth transition between the lead body 70 and the distal feature 714 to form a smooth tip junction 720 that will not snag cordae tendineae. The recess 730 can be located along a portion of the perimeter of the lead body 70 extending circumferentially for 45 degrees (range 20-90 degrees) such that the recess 730 is in axial alignment with the control opening 718 and located a recess spacing 732 of 1 mm (range zero to 3 mm) in a direction proximal to the control opening 718. Alternately, the recess 730 can extend around the entire perimeter of the lead body 70 at a recess spacing 732 and recess dimension 733 as described previously. The recess 730 can provide a protective recess dimension 733 of 0.100 inches (range 0.020-0.200 inches). The distal feature 714 can have a geometrical shape that is either symmetrical or asymmetrical with respect to the central axis 450 of the lead body 70 and distal feature 714. An asymmetric shape can be formed to mate with the lead body 70 within the recess 730 and form a smooth transition along the surface of the pacing lead 5 from the lead body 70 to the tip junction 720.

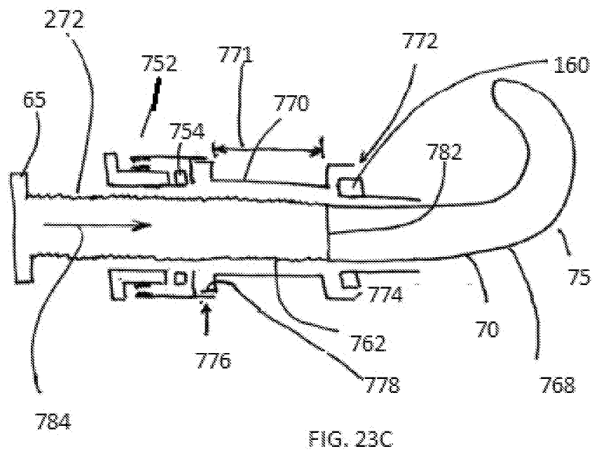

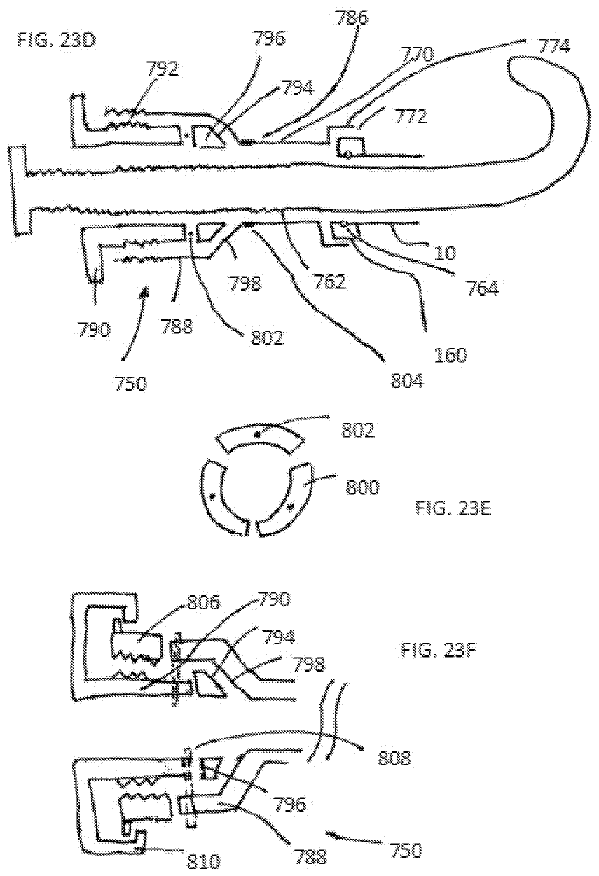

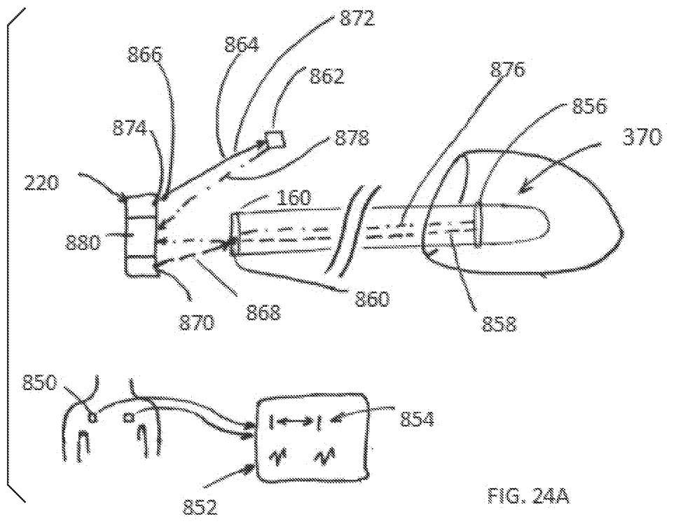

The importance of locking the lead body 70 of the pacing lead 5 with the introducer sheath 10 was described earlier in FIG. 10C. The locking together of these two components can ensure that the pacing lead 5 is not inadvertently moved relative to the endocardial surface of the heart resulting in loss of capture or sensing within the heart, potentially leading to serious patient sequellae. Several embodiments are presented in FIGS. 23A-23F for locking the pacing lead 5 to the introducer sheath 10. In FIG. 23A a locking assembly 750, such as a Tuohy-Borst (TB) connector of a TB component 752, commonly used in the medical device industry for forming a leak-tight seal between a catheter shaft (located within a lumen of the TB component 752) and an external sheath (such as an introducer sheath, for example), is connected to the sheath manifold 160 of the introducer sheath 10. The TB component 752 can apply a compressive force onto a ring seal 754 located between the TB body 756 and the screw 758. Tightening of the screw 758 via a thread mechanism 760 with the TB body 756 causes the ring seal 754 to compress via uniaxial compression and extend radially inwards and outwards and extend into contact with the lead body 70. A roughened or ribbed surface 762 can be placed onto the inner surface ring seal 754 that makes contact with the lead body 70; the ribbed surface 762 will enhance the friction between the ring seal 754 and the lead body 70 to prevent lateral movement of the lead body 70 relative to the TB component 752. The ring seal 754 must undergo a change in shape to preserve the volume of the ring seal 754 and hence the ring seal 754 moves outwards and inwards in a radial direction. The lead body 70 of the present embodiment has been formed with a ribbed surface 762 to allow a tight friction grip between the ring seal 754 and the ribbed surface of the lead body 70. The lead body 70 is thereby tightly grasped and fixed in place to prevent movement between the lead body 70 and the introducer sheath 10. A ribbed or roughened surface 762 for the lead body 70 can be formed by removing surface polymeric material, for example, from the lead body 70 with sandpaper, mechanical etching, chemical etching, or other roughening process. Alternately, the lead body 70 can be formed with a braided structure, wound with a coil wind, or formed with a structure that inherently is not smooth to give the lead body 70 a ribbed surface 762. The braided surface can be covered or coated with a polymeric film to encapsulate such a coil wind or braid and provide positional fixation to a coil wind or braid or provide a ribbed surface or frictional surface to the lead body 70. Such a ribbed or roughened surface will enhance the grip between the ring seal 754 and the ribbed or roughened surface 762 of the lead body 70 and ensure that the lead body 70 cannot move relative to the introducer sheath 10. The roughened surface 762 has a micro finish peaks and valleys; the ribbed or roughened surface 762 has an average peak-valley height of greater than 0.005 inches (range 0.002-0.010 inches).

The amount of length of lead body 70 in an axial direction that will extend into the patient's vasculature is often unknown precisely and the amount of lead body 70 that extends outside the body and outside of the introducer sheath 10 is also highly variable due to widely varying patient sizes, varying vasculature dimensions, and varying lead length requirements for each patient. The TB component 752 must be capable of providing a frictional lock between the roughened segment of the lead body 70 and the TB component 752 regardless of whether the patient requires a long lead body 70 length or a short lead body 70 length. The introducer sheath seal 764 located in the introducer sheath manifold 160 should also be capable of sealing to prevent leaks (between the introducer sheath 10 and the lead body 70) regardless of whether the patient requires a long lead body length or a short lead body length. A lead body smooth surface 768 may not provide an adequate frictional lock with the TB component 752. A lead body rough surface 762 may not provide a leak-tight seal with the introducer sheath manifold seal 764 and can traumatize the arterial wall 766 as shown in FIG. 23B.

To overcome potential concerns relating to having a roughened lead surface 762 contained within the introducer sheath seal 764 or within the patient's vasculature 766 or having a smooth surface 768 contained within the TB component 752, an extension tube 770 can be positioned between the introducer sheath manifold and the TB component 752. The extension tube 770 can have an extension-sheath attachment 772 between the extension tube distal connector 774 and the introducer sheath manifold 160 and can have an extension-TB attachment 776 between the extension tube proximal connector 778 and the TB component 752 as shown in FIG. 23C. The extension tube 770 allows the roughened surface 762 to be made in a lead body 70 region over a larger axial length in the lead proximal region thereby allowing the entire lead proximal region 272 of the lead body 70 (from the lead manifold 65 to the roughened junction 782 (i.e., where the roughened surface 762 meets the smooth surface 768 of the lead body 70) that is roughened to be used in all sizes of patients. In patients that require a small length for the lead body 70 the roughened junction 782 is located within the extension tube 770 within the TB component 752 (adjacent radially to the ring seal 754); in patients that require a longer lead body 70 the roughened surface 762 is located within the extension tube 770 with the roughened junction 782 near the introducer sheath manifold 160 as shown in FIG. 23C. The extension tube 770 has a length in the axial direction 784 equal to the difference in axial length required for the lead body 70 to accommodate various patient size requirements. In all patients, however, the smooth lead body 70 surface can traverse the vasculature, the introducer sheath 10, and the introducer sheath seal 764. The roughened surface 762 is able to be located radially adjacent to the ring seal 754 or other holding element 716 located on the TB component 752. In an alternate embodiment the extension tube 770 can be formed in a series of axial lengths 771 that accommodate the specific needs of a specific patient regarding the required lead body 70 axial length and placement of the TB component ring seal 754 radially adjacent to the lead body roughened surface 762 while placing the lead body smooth surface 768 into contact with the introducer sheath seal 764 located at the sheath manifold 160 and into contact with the vasculature.

The standard TB component 752 is normally intended to form a leak-free seal with a tubular shaft that is placed within its ring seal 754. The locking action between the pacing lead 5 and the introducer sheath 10 does not need to form a leak-free seal to form a lock that prevents movement between the lead body 70 and the introducer sheath 10. An embodiment for a locking assembly 750 is shown in FIGS. 23D-23F for forming a lock that prevents movement between the introducer sheath 10 and the lead body 70 but is not required to prevent fluid leakage between the lead body 70 and the locking assembly 750. In FIG. 23D is shown a locking assembly 750 that has an extension tube 770 that can be contiguously attached to the locking assembly 750 at the extension tube proximal end 786 and can have an extension tube distal connector 774 attached to the introducer sheath manifold 160 via an extension-sheath attachment 772. The locking assembly 750 is made up of a locking body 788 and a screw 790 that is similar to the TB component 752 described earlier. The screw 790 in this embodiment is able to move via screw thread mechanism 792 to cause a split ring bevel 794 of a split ring 796 to move adjacent to the body bevel 798 of the locking body 788. Tightening of the screw 790 causes the split ring 796 to move into contact with the roughened surface 762 of the lead body 70 as shown in FIG. 23D. The split ring 796 is formed from three ring segments 800 (see FIG. 23E) made of a resilient material such as silicone, polyurethane, or other rubber-like materials having good frictional properties when held under force against a polymeric material such as those used to make a lead body 70 for pacing leads or for angioplasty catheters. The split ring 796 can move via direct motion radially inward into contact with the lead body 70 rather than depending upon uniaxial compression forces created by three-dimensional volume conservation that are generated by a standard TB component 752. The split ring 796 is attached to the screw via three split ring attachments 802 that can be sliding attachments such that movement of the screw 790 to relieve the forces of friction from the split ring 796 to the lead body 70 will immediately allow the lead body 70 to have freedom of axial motion as the split ring 796 moves radially outwards from the lead body 70 to allow movement of the lead body 70 relative to the locking assembly 750 to be made for repositioning the lead body 70, for example. The split ring 796 of some embodiments may not provide a liquid tight seal like that provided by the standard TB component 752, but the split ring 796 provides for a tighter grasp of the lead body 70 than can be supplied by the TB component 752 described in FIG. 23C. The extension tube 770 can contain an extension-lock attachment 804 such that the extension tube 770 can be removed if desired for treatment of a patient requiring a longer length lead body 70.

FIG. 23F shows a further embodiment for the locking assembly 750 that has been described in FIG. 23D. In this embodiment, a locking nut 806 has been placed between the screw 790 and the lock body 788. The lock body 788 has been formed with a keyed slot 808 that keeps the locking body 788 circumferentially aligned with the screw 790. To tighten the screw 709 the locking nut 806 is rotated; the screw 709 which is attached to the nut 806 via a retainer 810 is forced to cause the split ring bevel 794 to move into contact with the body bevel 798 and cause the split ring 796 to make frictional contact with the roughened surface 762 of the lead body 70. Since the screw 790 is keyed with the locking body 788, there is no rotational motion against the split ring 796 as it is tightened; tightening of the nut 806 causes only linear motion of the screw 790 and causes the split ring 796 to move radially inward into frictional contact with the lead body 70 without rotation of the split ring 796.

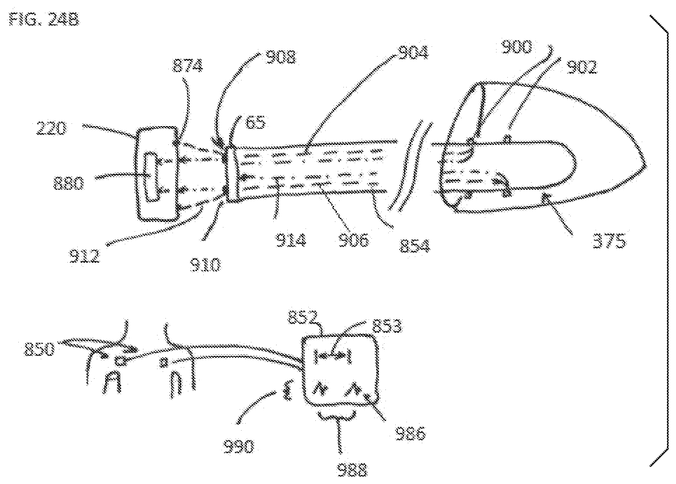

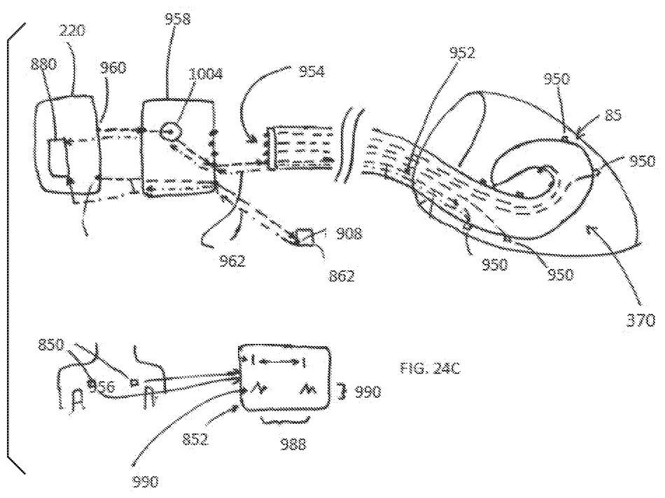

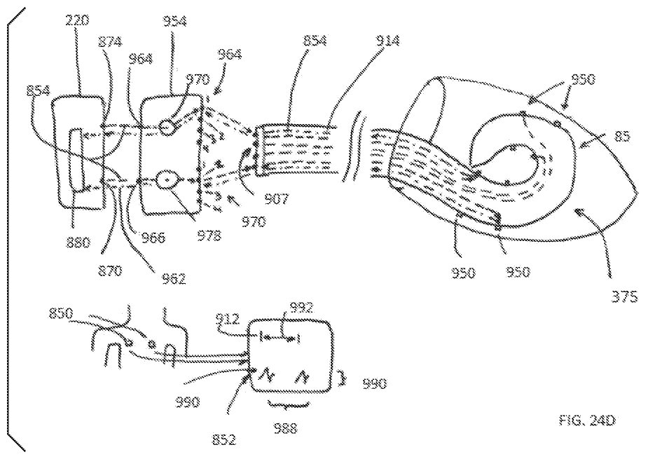

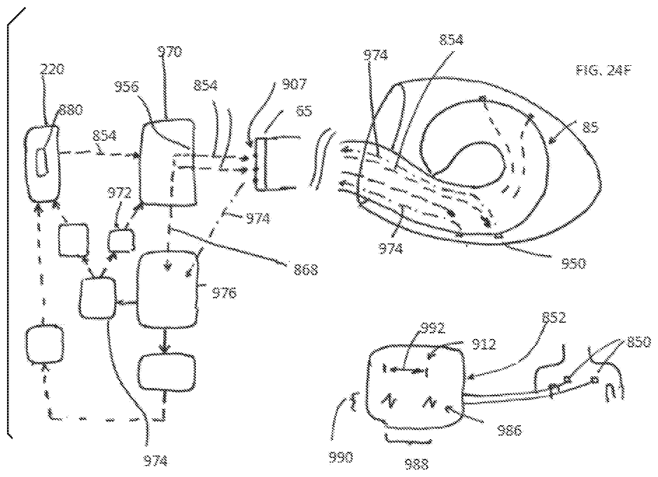

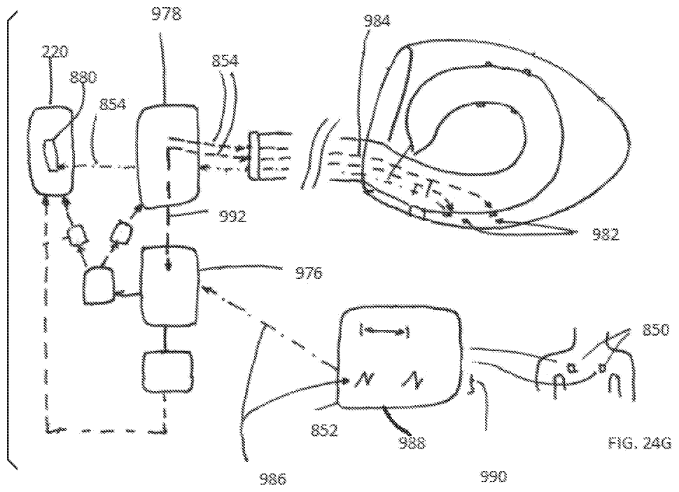

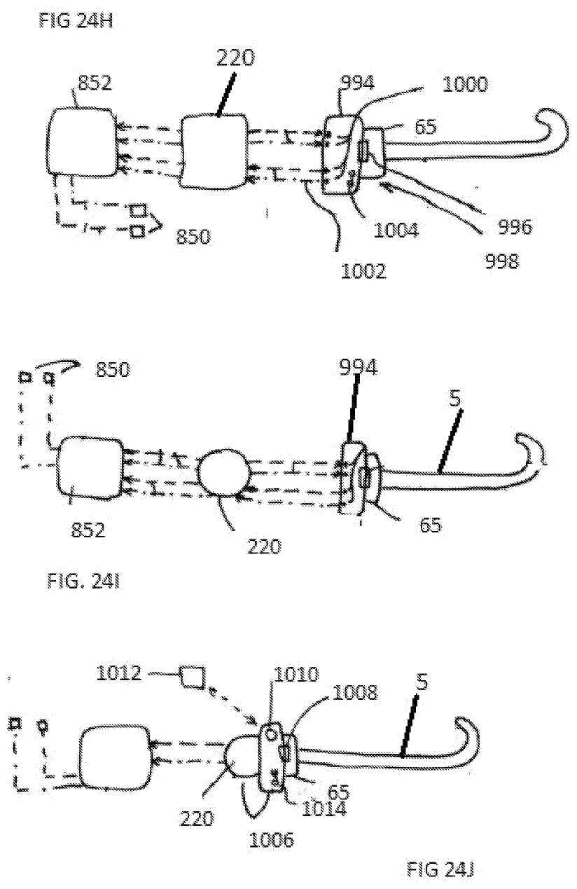

A unipolar and bipolar temporary pacing lead 5 have been described in FIGS. 2 and 11; however, their interface with the pulse generator 220 and method of use require further explanation. FIG. 24A shows how a standard single cathode unipolar electrode pacing lead or one cathode electrode of the unipolar pacing lead 370 of the present invention, for example, can be connected to the pulse generator 220. EKG electrodes 850 placed on the patient's anterior chest surface, for example can be used to provide an EKG signal of the heart that can be visualized by the operator on an EKG monitor 852 to determine if the pulse generator pulse signal 854 has attained capture by the heart. The cathode electrode 856 as shown in FIG. 24A can be a ring electrode located in the distal region of the catheter body; the cathode 856 is connected via a conduction wire 858 to a cathode connector 860 located on the lead manifold 160 that can be connected to one pole of the pulse generator 220. The anode 862 which can be located on the patient's body (such as the patient's back) is connected via a conduction wire 864 to an anode connector 866 that can be connected to another pole of the pulse generator 220. The pulse generator 220 sends out a generator cathode pulse signal 868 from the generator negative pole 870 to the cathode 856 and a generator anode pulse signal 872 from the generator positive pole 874 to the anode 862. The heart tissue will capture the generator pulse signal 872 and will provide a return cathode signal 876 or sensed signal from the myocardium from the cathode 856 and a return anode signal 878 or sensed signal from the anode 862 to a return signal receiver 880 located within the pulse generator 220. The return signal receiver 880 is able to receive and process a sensed signal from the myocardium and determine (for some pulse generator embodiments) if the pulse generator 220 should provide a subsequent pulse signal or inhibit subsequent delivery of a pulse signal. The same conduction wire 864 can be used to send the generator signal to the cathode 856 and provide the return sensed signal from the cathode 856 to the return signal receiver 880, for example. The return anode signal 878 and return cathode signal 876 is a sensed signal that is sensed from the myocardium when the lead electrode is in contact or near approximation with the myocardium. This sensed signal can be measured by the pulse generator receiver 880 of the pulse generator 220 to determine the rate of the sensed signal (beats/min) and the voltage amplitude of the sensed signal indicative of the ability or sensitivity of the lead electrode to sense the sensed signal.

The physician or operator is able to observe the pulse generator pulse signal rate and the myocardium conduction signal in response to the pulse signal on the EKG monitor 852 via signals obtained from EKG electrodes 850 placed onto the anterior surface of the patient's chest. The physician can compare the pulse generator pulse signal rate with the myocardial conduction rate (or heart rate) identified on the EKG 852 or electrocardiogram to determine if capture of the pulse signal has been achieved by the heart tissue. The pulse generator pulse signal 854 has a capture threshold (in mAmps) required to stimulate the myocardium and create a depolarization post-pacing spike that is sensed by an electrode of the temporary lead and provided as a return signal. The output setting of the pulse generator 220 is typically twice the capture threshold. The pulse generator 220 is set to a sensing threshold (mVolts) below which the pulse generator 220 will ignore a depolarization signal sent by the heart. The sensitivity value for the received depolarization signal is typically twice the sensing threshold. The myocardial conduction rate (heart rate) along with the EKG-sensed voltage amplitude of the heart systolic and diastolic peaks of the EKG-sensed myocardial signal can be observed by the physician using the EKG electrodes 850 on the patient's anterior chest surface or other appropriate surface.

A similar description is provided for the standard bipolar lead or to two electrodes located in the distal region of the bipolar temporary lead 325 of the present invention as shown in FIG. 24B. Here two ring electrodes, an anode 900 and a cathode 902, located in the distal region of the temporary pacing lead 5 are each connected via separate conduction wires 904, 906 to electrode connectors located at the lead manifold 65; an anode conduction wire 904 connects to an anode connector 908 and a cathode conduction wire 906 connects to a cathode connector 910. A PG pulse signal 912 is sent out from the pulse generator 220 to the anode 900 and cathode 902 and a return signal 914 that is sensing the myocardial conduction is returned from the anode 900 and cathode 902 to the pulse generator 220. The same conduction wire 906 for delivery of the generator cathode signal to the cathode 902 can be used to provide the return cathode signal 914 from that cathode 902 during the time period between the pulse signals.

EKG electrodes 850 placed on the patient's anterior chest surface can be used to provide visualization of the heart conduction signal as viewed on the EKG monitor 852. The heart electrical conduction rate in comparison to the pulse generator pulse rate can be visualized by the physician in order to determine if capture of the pulse generator signal by the myocardium has been attained.

The unipolar temporary pacing lead 5 of the present invention has a multiplicity of electrode sites including cathode sites as described earlier in one embodiment in FIG. 2 and presently described in FIG. 24C. The present invention has a multiplicity of electrode sites 950 to ensure that at least one of the sites 950 located in the distal region of the pacing lead 5 is making full contact with the endocardial surface such that capture of the PG pulse signal by the myocardium is maintained in a stable manner. If one electrode site 950 is inadvertently moved such that capture is lost, then a neighboring electrode site 950 can be manipulated manually by the physician (such as by adjusting the push-pull element 700 or control fiber 585 as shown in FIGS. 21A-21F, for example) into contact with the endocardial surface without requiring fluoroscopic guidance and preferably also without requiring echocardiographic guidance. Each electrode site 950 is connected individually via a separate insulated conduction wire 952 to a separate electrode connector 954 located at the lead manifold 65. The anode 862 for the unipolar lead embodiment can be connected to the pulse generator positive pole 956 as shown in FIG. 24C and described earlier in FIGS. 10A-10C; the anode 862 for a temporary lead can be located on the patient's body (such as the patient's back, for example). The anode for a fully implantable pacing lead can be the metal outer container of the pulse generator 220.

When initially placing the unipolar temporary pacing lead 370, the physician attaches a pulse generator 220 to the cathode connector 954 and anode connector 908. The cathode 950 can be a multiplicity of ring electrodes, for example, that form the cathode sites located in the distal region of the lead body 70 as shown in FIG. 24C. Located between the cathode connectors 954 and the pulse generator 220 of this embodiment is a manual switch box 958. The manual switch box 958 takes the generator cathode signal coming from the negative pole 960 of the pulse generator 220 and directs it to one of four cathode connectors 954 that form individual continuity with one of four cathode sites 950 (range 2-20 sites), cathode 1, cathode 2, cathode 3 or cathode 4. As shown in FIG. 24C the generator cathode signal can be directed via cathode connector 1, for example, to cathode 1. The manual switch box 958 also directs the return cathode signal 962 which is the lead sensed signal generated from the myocardial contraction in response to the pulsed signal from cathode 1 back to the return signal receiver of the pulse generator 220. The return signal receiver 880 in some embodiments can measure and record the rate of the return signal (i.e., the lead sensed signal which is indicative of the number of myocardial contractions per minute) and the voltage amplitude of the lead sensed signal. The return signal receiver 880 in some embodiments can determine if the myocardium is able to provide its own intrinsic pacing signal and can inhibit the delivery of the PG pulse signal. The manual switch box 958 takes the generator anode signal from the generator positive pole 956 and directs it to the anode 862. The return signal from the anode 862 can also be directed to the return signal receiver 880 of the pulse generator 220 to identify the rate and voltage magnitude of the sensed signal.

The physician or operator can determine if capture has been attained via visualization of the EKG monitor 852. EKG-sensed myocardial conduction signals from the EKG electrodes 850 provide the operator with the myocardial conduction rate (or heart rate). Comparison of the EKG-sensed myocardial conduction rate with the PG pulse signal rate allows the operator to determine if capture has been attained. The physician first sets the pulse rate of the pulse generator 220 to a pulse rate that is higher than the intrinsic heart rate of the patient. The pulse current of the pulse generator 220 is set to a value (mAmps) that will ensure capture of the myocardium. The physician can observe the EKG-sensed myocardial conduction signal to assess whether the EKG-sensed myocardial conduction rate is equal to the PG pulse signal rate indicating that capture of the pulsed signal by the myocardium has occurred. If capture has been attained, the pulse signal current from the pulse generator 220 is then toggled downwards until capture is lost and the heart rate as observed via EKG has returned to the slower intrinsic heart rate. The lowest pulse generator pulse current that provides capture is then obtained; this is the threshold current for the initial electrode pair. The EKG-sensed voltage amplitude of the EKG-sensed myocardial conduction signal is also recorded along with the pulse signal threshold current. The physician then adjusts the manual switch box 958 to direct a PG pulse signal from the pulse generator 220 to a second electrode pair. A similar assessment is made of the threshold current for the pulsed signal and the EKG-sensed voltage amplitude of the return signal or sensed signal for the second electrode pair. Similar assessment is made for each electrode pair of the present temporary lead. The electrode pair having the lowest threshold current and having the greatest EKG-sensed voltage amplitude is chosen by the physician for pacing of the myocardium. The pulse signal current is set to approximately twice the threshold current for pacing the heart.

The bipolar temporary pacing lead 5 of the present invention has a multiplicity of electrode sites located in the lead distal region 85 of the pacing lead 5 described in FIG. 24D. Each electrode site 950 has a separate insulated conduction wire 952 extending back to a separate electrode connection at the lead manifold 65. The present invention has a multiplicity of electrode sites 950 located adjacent to each other in the distal region to ensure that at least one electrode pair of two neighboring electrodes or of two separate electrodes in the lead distal region 85 are making full contact with the endocardial surface such that capture is maintained in a stable manner Each of the electrode sites 950 can function either as a cathode or an anode depending upon the electrode connection to the generator anode signal or generator cathode signal of the pulse generator 220. If the catheter body is inadvertently moved such that one electrode pair is no longer in contact with the myocardium and capture is lost, then a neighboring electrode pair can be manipulated manually by the physician (including adjusting the push-pull element 700 or control fiber 585 as shown in FIGS. 21A-21F) into contact with the endocardial surface without requiring fluoroscopic guidance or echocardiographic guidance.

Upon initially placing the bipolar temporary pacing lead 375, the physician attaches a pulse generator positive pole 874 to the switch box anode input receptacle 964 and attaches the pulse generator negative pole 870 to the switch box cathode input receptacle 966. The manual switch box 958 takes the generator cathode signal coming from the generator negative pole 870 of the pulse generator 220 and directs it via the switch box anode output receptacle 968 and cathode output receptacle 970 to one of four electrode sites 950 (range 2-20 sites), electrode 1, electrode 2, electrode 3 or electrode 4. As shown in FIG. 24D the pulse generator cathode pulse signal is being directed, for example, to electrode 2. The manual switch box 958 also directs the return signal or lead sensed signal generated by the myocardium from electrode 2 back to the return signal receiver of the pulse generator 220. A single conduction wire 952 can be used to provide the generated cathode signal to electrode 2 as the return cathode signal from electrode 2, the return signal being sensed and delivered during the time period between sending pulse signals from the pulse generator 220. As shown in FIG. 24D the generator anode signal is being directed from the generator positive pole 874 to electrode 1, for example. The manual switch box 958 also can direct the return anode signal back from electrode 1 to the return signal receiver of the pulse generator 220. The return signal receiver 880 is able to sense the rate of lead sensed signals (indicative of myocardial contractions/minute) and also sense the voltage amplitude of the lead sensed signal.

EKG electrodes 850 attached to the patient's chest provide EKG-sensed myocardial conduction signals indicative of heart rate along with EKG-sensed myocardial voltage amplitudes directly indicative of the contact of the lead electrodes with the myocardium. The physician or operator can determine if capture has been attained by examining pulse rate from the pulse generator 220 and myocardial heart rate as visualized from the EKG monitor 852. It is necessary to identify the lowest value of the PC pulse signal current that is able to effect capture by the myocardium. If capture is attained with a specific electrode pair, the current (mAmps) of the pulse generator pulse signal 854 is toggled downwards until capture is lost. The physician records the lowest PG pulse signal current that is able to provide capture by the myocardium (the threshold current). The physician or operator can then manually moves the manual cathode switch 968 or anode switch 970 to direct the generator cathode signal to two different electrodes that make up a different electrode pair than the initial electrode pair, and again determine the threshold current level for which capture is attained for the second electrode pair. The physician can then select the electrode pair having the lowest threshold current and having the highest EKG-sensed myocardial voltage amplitude indicative of the optimal electrode pair to provide consistent myocardial pacing.

The present invention can comprise a manual switch box 958 as described in FIGS. 24C and 24D; alternately, the switch box 958 can be automated such that the choice of the electrode pair is performed automatically as shown in FIG. 24F. In this embodiment a lead automatic switch box 970 is used to choose an electrode pair 972 based on lead sensed signals that are sensed by the lead electrode pair. As discussed in previous embodiments a pulse generator 220 provides a pulse generator signal pulse 868 to the lead automatic switch box 970 which directs the signal to an electrode pair 972. The pulse generator signal 868 is provided at a higher rate than the intrinsic heart rate and at a PG pulse signal current (mAmp) that is high enough to attain capture by the myocardium. The electrode pair 972 can be two electrodes 950 located in the distal region of the lead 5 (i.e., for a bipolar lead) or the electrode pair can include one external electrode (located on the patient's back, for example) and one electrode located on the lead distal region 85. Following delivery of the signal pulse to the myocardium the electrode pair 972 serves to sense the myocardial electrical conduction and directs a lead sensed myocardial signal 974 back to the lead manifold 65 and to a comparator 976. The comparator 976 measures the pulse rate of the pulse generator pulse signal 854 and compares it with the lead sensed myocardial signal rate 974 (indicative of myocardial conduction rate or heart rate) as measured by the comparator 976. The comparator 976 also measures the lead sensed voltage amplitude of the lead sensed myocardial signal to determine the amplitude of the sensing signal. A higher lead sensed voltage amplitude is indicative of an improved contact of the lead electrode pair with the myocardium and an improved likelihood for consistent pacing of the myocardium. If the lead sensed myocardial signal rate is the same as the PG pulse signal rate, then capture has occurred and it is then necessary to determine the threshold current or lowest current in the pulse generator pulse signal 854 that will provide capture of the myocardium for that electrode pair. A low threshold current reflects improved contact of the electrode pair with the myocardium and improved capture of the myocardium of the pulse generator pulse signal 854.

To identify the threshold, current the comparator signals the pulse generator 220 to toggle down the pulse signal current. As the pulse current is reduced the comparator 976 is continually monitoring the lead sensed myocardial signal rate and comparing it to the PG pulse signal rate. When the lead sensed myocardial signal rate returns to the intrinsic rate of the non-paced heart (that is slower than the paced rate), capture has been lost and the lowest pulse generator pulse signal current that was able to maintain capture is retained by the comparator 976 and stored as the threshold current for that electrode pair. The comparator 976 also retains the lead sensed voltage amplitude at the pulse signal threshold current for that initial electrode pair. The comparator 976 sends a signal to the lead automatic switch box 970 to switch to a second electrode pair 972. The comparator 976 also signals the pulse generator 220 to send a pulse generator pulse signal 854 to the lead automatic switch box; the PG pulse signal is of a large pulse current to cause capture using a second electrode pair 972. In a manner similar to that described for the initial electrode pair, the threshold current for the PG pulse signal and lead sensed myocardial voltage amplitude is measured and recorded by the comparator 976 for the second electrode pair 972. Examination is made of each electrode pair 972 for assessment of PG pulse signal threshold current and lead sensed myocardial voltage amplitude, the comparator 972 optimally chooses an electrode pair having the lowest PG pulse signal threshold current and having the largest lead sensed myocardial voltage amplitude. The operating current provided to the selected electrode pair for providing pacing to the myocardium is approximately twice the PG pulse signal threshold current.

The physician is able to monitor the steps taken by the lead automatic switch box 970. EKG electrodes 850 place onto the anterior skin of the patient's chest can be connected to an EKG monitor 852 which provides an examination of the full EKG electrical waveform of the heart. The physician can view the heart rate and can view the magnitude of the voltage amplitude for the electrode pair that is being chosen.

An alternate embodiment for an automatic switch box includes using the EKG electrodes 850 placed on the patient's chest to automatically determine which electrode pair is best suited to provide consistent capture of the myocardium. The EKG automated switch box 978 of this embodiment is shown in FIG. 24G. The EKG automatic switch box 978 direct a PG pulse signal 980 to an initial electrode pair 982 associated with the pacing lead 5 of the present invention. The pulse signal current 984 is set large enough to ensure that capture of the myocardium is attained. Instead of the comparator 976 receiving a lead sensed myocardial signal from the lead electrode pair, an EKG-sensed myocardial signal 986 is provided to the comparator 976. The EKG-sensed myocardial signal 986 provides information including the heart rate 988 and the EKG-sensed myocardial voltage amplitude 990 of the EKG-sensed myocardial conduction signal 986. The comparator 976 compares the EKG-sensed myocardial conduction rate 988 with the pulse generator pulse signal rate 992 to determine if capture has been attained. If the EKG-sensed myocardial rate 988 is equal to the PG pulse signal rate 992, then capture has occurred, and steps are taken to identify the PG pulse signal threshold current for that initial electrode pair 982. The comparator 976 also stores the EKG-sensed myocardial voltage amplitude 990 of the EKG-sensed myocardial conduction signal 986.

The pulse signal current is toggled down as described in the embodiment of FIG. 24G to identify the threshold current that still provides for capture of the signal by the myocardium. The comparator 976 stores the PG pulse signal threshold current along with the EKG-sensed myocardial voltage amplitude of the sensed signal. Each electrode pair 982 is examined in a manner similar to that described in the embodiment of FIG. 24G. The optimal electrode pair is chosen to have the lowest PG pulse signal threshold current along with the highest EKG-sensed myocardial voltage amplitude.

The automatic switch box 978 can be miniaturized and made into a component of the lead manifold to form a switch-manifold (SM) component thereby simplifying the system as shown in FIG. 24H. The lead manifold 65 can connect to the switch box 994 via a manifold-switch connector 996 to provide electrical coupling between the electrode sites of the pacing lead and the switch box. The simplified system provides improved ambulation for the patient receiving electrical stimulation of the heart via the temporary lead. The switch manifold (SM) component 998 has a signal connector 1000 that allows the SM component 998 to be connected to the pulse generator 220 and to provide a return anode signal 1002 that can be monitored for signal capture. The SM component 998 can be connected to the unipolar pacing lead of the present invention or the bipolar pacing lead of the present invention as described in FIG. 24G. For a unipolar or bipolar lead, the cathode electrode is one of the electrode sites located in the distal region of the pacing lead. For a bipolar lead, the anode is another of the electrode sites located in the distal region of the pacing lead other than the electrode site chosen for the cathode. For a unipolar lead, the anode is a separate remote electrode located on the patient's skin, on the introducer sheath, or other location for a patch anode electrode. The SM component 998 has a mode switch 1004 to indicate whether a unipolar pacing lead or a bipolar pacing lead is being placed into the chamber of the heart and thereby indicate to the switch box portion of the SM component the sequence to follow for choosing an electrode pair, determining if capture has been attained, if capture is not attained, then the electrode pair is altered until efficient signal capture has been attained from the pulse generator 220 to the myocardium.

The pulse generator 220 is currently a bulky and cumbersome piece of equipment in the cardiology suite and as a system component that is transferred along with the patient into the intensive care room or recovery room. Therefore, a further aspect of the present invention to miniaturize the pulse generator 220 although still supply the pulse generator 220 as a reusable piece of equipment as shown in FIG. 24I. The pulse generator 220 can be operated via battery power or via wall outlet and can be easily moved along with the patient without causing small movements of the electrodes 850 relative to the heart endocardial surface and loss of capture.

A further embodiment for the temporary pacing system of the present invention is to miniaturize the pulse generator 220 to a mini-generator and combine the mini-generator with the switch box and provide these components as a single generator-switch-manifold (GSM) 1006 component that has been combined with the lead manifold 65 as shown in FIG. 24J. The lead manifold 65 can connect to the switch box and pulse generator 220 via a manifold-switch connector 1008 to provide electrical coupling between the electrode sites of the pacing lead 5 and the switch box and the pulse generator 220. This automatic generator-switch-manifold embodiment 1006 provides a smaller, more convenient, and safer system for the patient that is required to use the temporary pacing lead for a period of several days without encountering complications associated with lead movement due to the current cumbersome system components. The automatic generator-switch-manifold and temporary lead can be provided as a single disposable entity that can be powered by battery power and is able to provide convenient mobilization to the patient until a more permanent therapy for the patient has been identified. The GSM 1006 can be placed comfortably onto the anterior chest surface of the patient for a period of several days while the lead extends percutaneously via an introducer sheath into the subclavian vein and traverses to the left ventricular chamber to provide temporary pacing.

The GSM component 1006 can alternately be implanted into a subcutaneous pocket under the skin of the patient's chest thereby making the GSM component 1006 and pacing lead 5 a fully implantable temporary lead system. In this embodiment the containment vessel for the pulse generator 220 can serve as a remote electrode or an anode electrode for a unipolar temporary pacing catheter system. The GSM 1006 is fitted with an RF transmitter/receiver 1010 that is able to receive signals from an operator via an RF controller 1012. If signal capture has been lost, an operator would be able to identify and establish a new electrode pair that reestablishes capture of the pulse generator signal by the myocardium of the heart via radiofrequency communication or via remote telemetry.

In a significant percentage of TAVR patients it has been found that permanent pacing systems are not needed after approximately 5 weeks (range 3-8 weeks) post implant. Permanent pacing leads are often difficult to remove after several weeks of implant due to the presence of tines or other mechanisms used to provide improved fixation of the permanent lead with the endocardial surface of the heart chamber; the fully implantable temporary lead of the present invention overcomes these objections by eliminating fixation mechanism in the distal region of the temporary pacing lead.

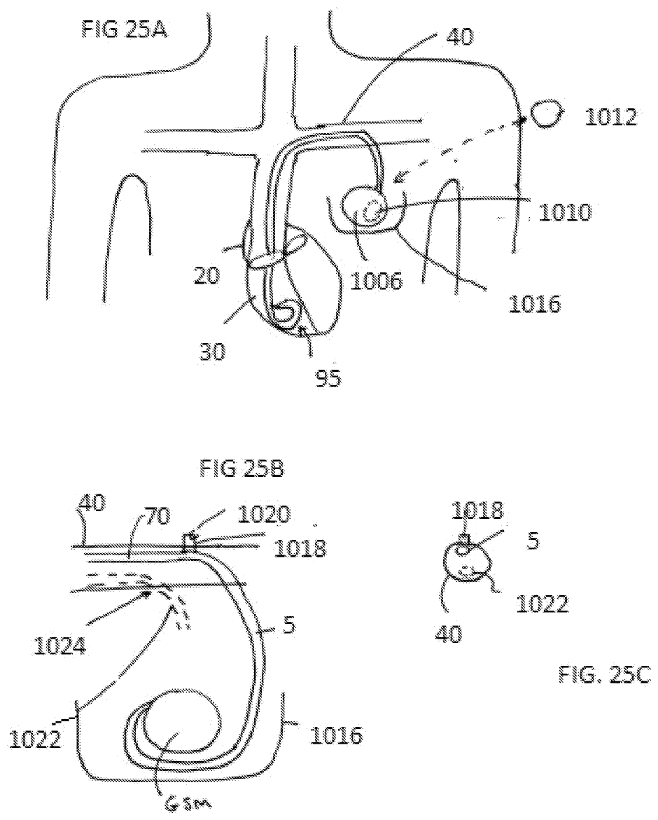

One embodiment for the temporary pacing lead of the present invention is shown in FIGS. 24J and 25A-25C. The pacing lead 5 as shown in FIG. 24J has a generator-switch-manifold component 1006 (i.e., GSM component) that is small enough to be implanted; therefore, this temporary lead and the GSM component together provide a fully implantable GSM-temporary lead system. The GSM component 1006 has a mode switch 1014 that allows the operator to choose between a bipolar and unipolar lead. The GSM 1006 will automatically select an electrode pair that provides capture of the pulse generator signal by the heart. The GSM 1006 will also allow the electrode pair to be adjusted at a later time after it has been implanted via the RF transmitter/receiver 1010 that communicates between the remote RF controller 1012 and the RF transmitter/receiver 1010 of the GSM component 1006.

FIG. 25A shows the GSM-temporary lead system 1006 in an implanted configuration. The temporary lead 5 enters the left subclavian vein 40 and is directed to the right ventricular chamber 30. The temporary lead 5 forms a closed loop 95 as described in embodiments described in the present specification. The present temporary lead 5 does not have tines or other fixation mechanisms and therefore is easily removed after several weeks of implantation. The present temporary lead 5 provides definite contact of the electrode sites with the myocardial surface due to the formation of the closed loop 95 that contacts two walls of the ventricular chamber near the apex of the heart. The proximal lead body 70 is secured to the subcutaneous tissues near the access site for entry of the lead body 70 into the subclavian vein 40 via a sewing tab 1018 that is attached to surrounding tissues with sutures 1020. The temporary pacing lead 5 is a low-cost lead that provides for ventricular pacing, ventricular sensing, inhibition of signaling if the native signal is functioning, and rate responsive (i.e., VVIR), for example. The GSM component 1006 is placed into a subcutaneous pocket 1016 in the patient's chest. Any redundant lead body is coiled into a lead body coil and placed into the subcutaneous pocket 1016 along with the GSM component 1006.

The GSM-temporary lead provides a cost-effective means for providing temporary pacing for a period of 5 weeks post TAVR. After the 5-week period, the patient is evaluated to identify if there remains a need for continued pacing. If normal sinus rhythm and normal signal conduction is observed, the temporary pacing lead 5 and GSM component 1006 can be easily removed. If it is determined that a permanent pacing lead 1022 needs to be implanted, then a permanent lead access site 1024 located laterally a few centimeters away and on an opposing side of the subclavian vein 40 can provide an access site for placement of a new permanent lead and new pulse generator 220 as shown in FIGS. 25B and 25C. The permanent pulse generator 220 can be placed into the previously formed subcutaneous pocket used for the GSM component or a new pocket can be formed nearby.

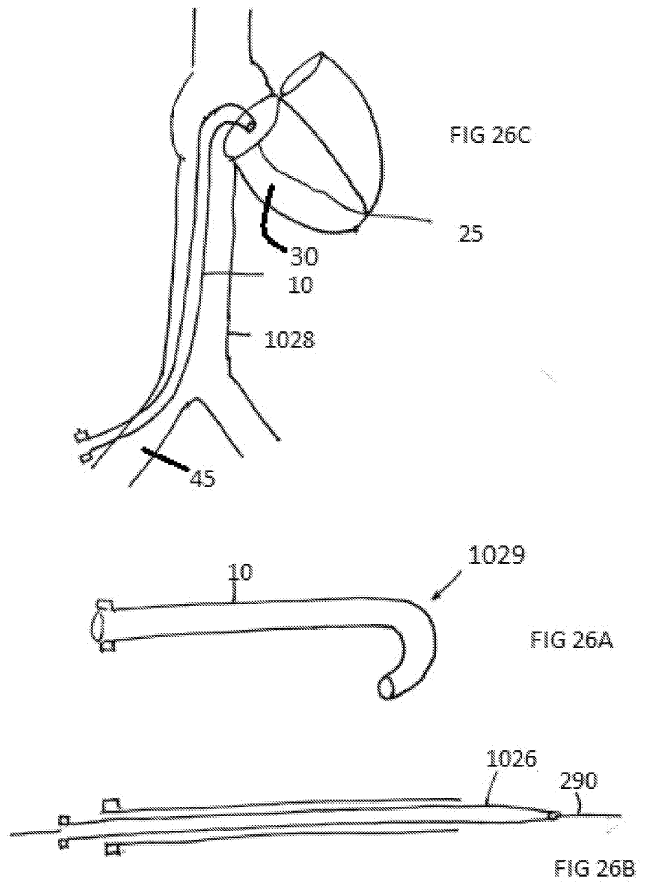

The temporary lead system shown in FIGS. 24H and 24J can be used for temporary pacing for a few days or up to one week to treat cardiac arrhythmias of all types with the pacing lead entering venous access sites such as the femoral vein, internal jugular vein, and subclavian vein through an introducer sheath. For cases where rapid and reliable entry into the venous vasculature is needed, the femoral vein is an acceptable access site. As shown in FIGS. 26A and 26B an introducer sheath 10 with a precurved introducer distal end 1029 is fitted with a dilator 1026 to cause the precurved distal end 75 to straighten for percutaneous entry into the femoral vein 45 and traversing the vasculature. The introducer sheath 10 and dilator 1026 assembly is entered into the femoral vein 45 and advanced over a guidewire 290 through the inferior vena cava 1028 and into the right atrium 20 where the dilator 1026 is withdrawn allowing the precurved distal region of the introducer sheath 10 to be directed toward the tricuspid valve 25 as shown in FIG. 26C.

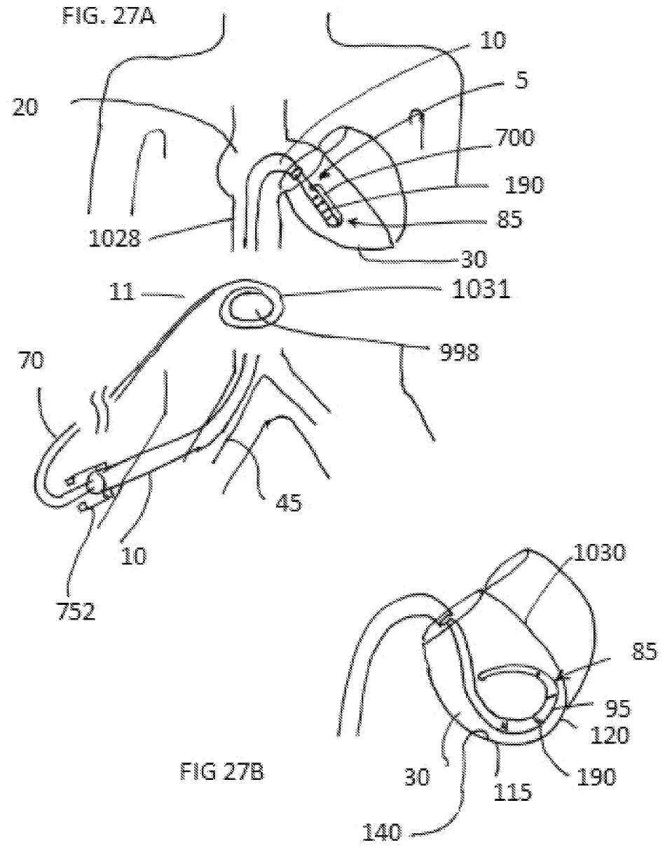

FIG. 27A shows the pacing lead 5 of the present invention being placed within the body via an introducer sheath 10 having a femoral vein 45 access. The lead distal region 85 has a linear configuration as the lead distal region 85 enters the tricuspid valve 25 annulus and into the right ventricular chamber 30 of the heart 35. The push-pull element 700 or control fiber 585 extends from the lead distal end 75 to the control opening 718 with the push-pull element 700 or control fiber 585 not placed under tension and the curled shaft 80 has a generally linear of slightly curved configuration. Further advancement of the pacing lead 5 within the introducer sheath 10 places the lead distal region 85 further into the right ventricle 30 near the apex 120 of the heart 35. Within the right ventricle 30 tension is applied to the push-pull element 700 to bring the lead distal end 75 into contact with the control opening 718 to form the lead distal region 85 into a closed loop 95 as shown in FIG. 27B. The electrode sites 190 are placed into contact with the endocardial surface 140 of the ventricular septum 1030 and the lateral wall 115 of the right ventricle 30.

The proximal lead body 70 extends out of the introducer sheath 10 and out of the patient's body and is directed toward the patient's anterior abdominal surface. The lead body 70 exits proximally through the introducer sheath manifold 160 and passes through a Touhy-Borst (TB) component 752 that is attached to the introducer sheath manifold 160. The TB component 752 serves to lock the proximal lead body 70 relative to the introducer sheath 10 and prevents migration of the lead distal region 85 located adjacent to the endocardial surface 140. The proximal lead body 70 typically has excess or redundant length that is formed into a coiled lead body 1031. The lead manifold 65 is connected to the switch box 958 via a manifold-switch connector 996; the lead manifold 65 and switch box 958 are electrically coupled to form the SM component 998 which is located on the anterior surface of the abdomen as shown in FIG. 27A. The pulse generator 220 can be coupled to the SM component as a single GSM or as a separate component.

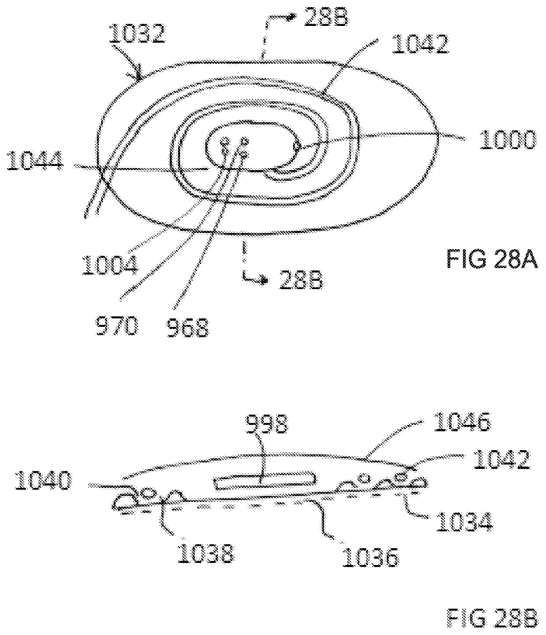

FIGS. 28A and 28B show a close up of one embodiment of a redundant lead holder 1032 that can be placed onto the patient's anterior abdomen to hold excess length of lead body 70 and also hold the SM component 998 (i.e., switch box 958 and lead manifold 65) or a GSM component 1006. The redundant lead holder 1032 has an adhesive 1034 on a lower surface 1036 to adhere the redundant lead holder 1032 to the patient's skin. A spiral groove 1038 is located on the upper surface 1040 to allow redundant lead body 1042 to be spirally wound within the groove 1038 and attach to the SM component 998 located in the central space 1044 in the center of the spiral wind. The upper surface 1040 of the redundant lead holder 1032 has a sticky surface to allow the redundant lead body 1042 to adhere to the spiral groove. A signal connector located on the SM component 998 can be connected to the pulse generator 220. A clear plastic cover 1046 can be placed over the SM component 998 to allow manipulation of the mode switch 1004 for selection of unipolar or bipolar lead. Also, an anode switch 970 and cathode switch 968 can be manipulated as shown in FIG. 24F to select an electrode site pair that provides for signal capture for the case that the switch box is a manual switch box.

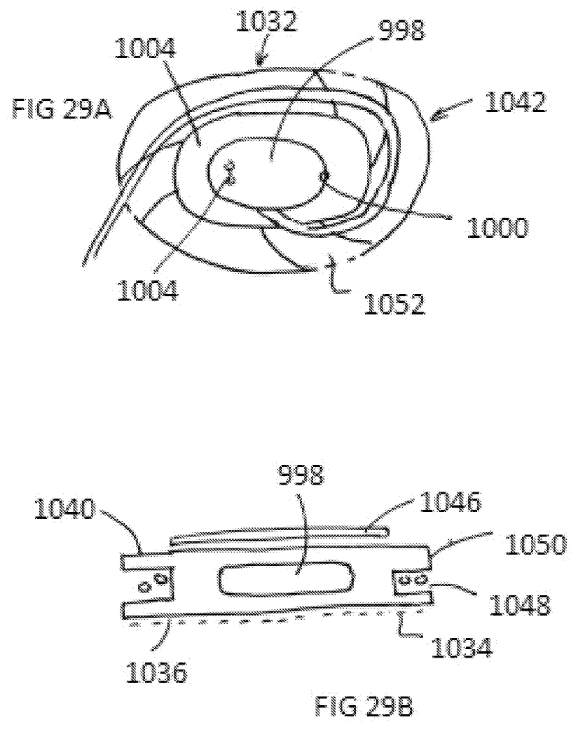

An alternate configuration for the redundant lead holder is shown in FIGS. 29A and 29B. This embodiment of the redundant lead holder 1032 has an adhesive 1034 on the lower surface 1036 to provides adhesion of the redundant lead holder 1032 to the anterior chest surface of the patient. The redundant lead body 1042 is wound around a slot 1048 in a spool 1050. Frontal openings 1052 provide passage of the lead body 1042 into the central space 1004. The SM component 998 is located in the central space 1004 and a clear plastic cover 1046 is located on the upper surface 1040 over the SM component 998 through which the mode switch 1004 and other features of the GM component 1006 can be manipulated by the operator.

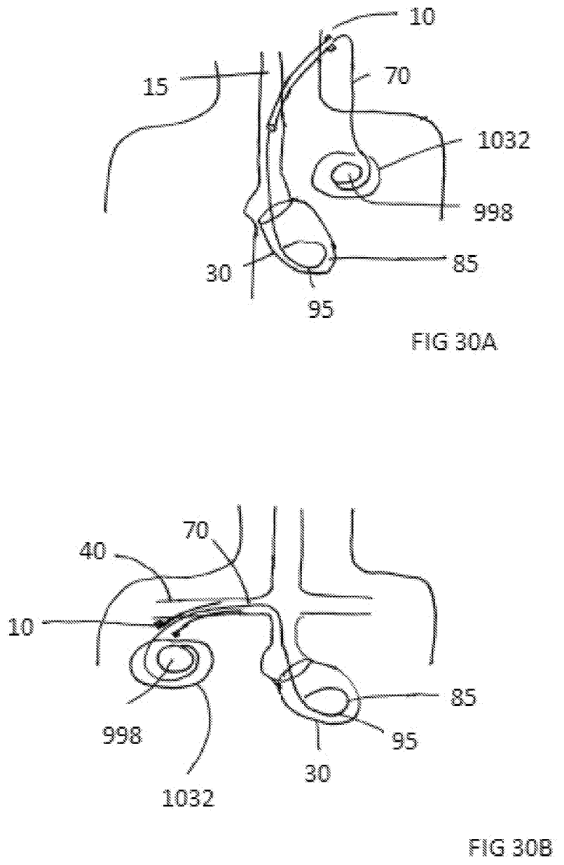

The temporary lead of the present embodiment can be placed for a few days via an access site in the internal jugular vein or subclavian vein with the lead manifold, switch box, and pulse generator 220 externalized outside of the body. The pacing lead system can include the manual switch box 958, as shown in FIG. 24D or the automatic switch box 970 as shown in FIGS. 24G and 24H. Placement of the temporary lead of the present invention via the internal jugular vein 15 can be performed as shown in FIG. 30A or via the subclavian vein 40 as shown in FIG. 30B. An introducer sheath 10 is placed into the internal jugular vein 15 or the subclavian vein 40 using standard techniques. The lead 5 enters an introducer sheath 10 and is advanced into the right ventricular chamber 30 where the lead distal region 85 is formed into a closed loop 95 as described previously in FIG. 27B for the femoral access approach. Redundant lead body 1042 is coiled or wound onto a redundant lead body holder 1032 as described earlier and the lead manifold 160 is connected to the switch box 958 via a manifold-switch connector 1008 (see FIG. 24G). The SM component 998 is placed into the central space 1004 of the redundant lead body holder 1032 which is held via an adhesive 1034 on the lower surface 1036 of the lead body holder to the patient's skin on the anterior chest surface.

Any version of any component or method step of the invention may be used with any other component or method step of the invention. The elements described herein can be used in any combination whether explicitly described or not.

All combinations of method steps as used herein can be performed in any order, unless otherwise specified or clearly implied to the contrary by the context in which the referenced combination is made. Also, if a method is described for use with a control fiber that can be placed only under tension and using the stored elastic energy of the shaft to effect a return to a linear shape as described in the embodiment of FIGS. 21A-21C, it is understood that the embodiment described in FIGS. 21D-21E can also be used for that method wherein the tension-compression member 702 is able to provide the compression forces in the push-pull element 700 that cause the lead distal region 85 to make a more forceful contact with the myocardial wall or return the lead distal region 85 to a more linear shape.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference in their entirety to the same extent as if each individual reference were specifically and individually indicated as being incorporated by reference. In case of conflict between the present disclosure and the incorporated references, the present disclosure controls.

The devices, methods, compounds and compositions of the present invention can comprise, consist of, or consist essentially of the essential elements and limitations described herein, as well as any additional or optional steps, ingredients, components, or limitations described herein or otherwise useful in the art.

While this invention may be embodied in many forms, what is described in detail herein is a specific preferred embodiment of the invention. The present disclosure is an exemplification of the principles of the invention is not intended to limit the invention to the particular embodiments illustrated. It is to be understood that this invention is not limited to the particular examples, process steps, and materials disclosed herein as such process steps and materials may vary somewhat. It is also understood that the terminology used herein is used for the purpose of describing particular embodiments only and is not intended to be limiting since the scope of the present invention will be limited to only the appended claims and equivalents thereof

The invention claimed is:

1. A pacing lead for temporary atraumatic placement via transvascular access on an endocardial surface of a heart chamber of an animal body part to deliver an electrical signal comprising:
a. a lead manifold located outside the animal body; and
b. a pacing lead body connected to the lead manifold, the pacing lead body having a proximal end and a distal end, wherein the pacing lead body comprises a curled shaft at the distal end region of said pacing lead body, wherein the curled shaft has a distal end and a proximal end for temporary placement of the curled shaft against the endocardial surface, wherein the curled shaft further comprises a plurality of electrode sites, which electrode sites are connected via electrical continuity such that at least one of the plurality of electrode sites is adapted temporarily connect to the endocardial surface, wherein each of the plurality of electrode sites is connected to an individual conduction wire, wherein each of the plurality of conduction wires extends along the pacing lead body to connect to an individual electrode connector on the lead manifold, wherein each of the plurality of electrode connectors is individually connected via the individual electrode conduction wire to a plurality of electrode receptacles of a switch box, wherein the switch box is adapted to receive generator signals from a pulse generator and direct the electrode generator signal to specific electrode sites.

2. The pacing lead of claim 1 wherein the switch box is electrically coupled and physically combined with the manifold thereby forming a switch-manifold component that is attached to the proximal end of the lead body.

3. The pacing lead of claim 2 wherein the pulse generator is electrically coupled and physically combined with the switch-manifold component thereby forming a generator-switch-manifold component which is attached to the proximal end of the lead body.

4. The pacing lead of claim 3 wherein the generator-switch-manifold component in combination with the lead body comprises the pacing lead which is configured to be temporarily implanted subcutaneously and within the heart chamber of the animal body.

5. The pacing lead of claim 1 further comprising a push-pull element connected to the lead body distal end, wherein the push-pull element traverses externally to the lead body distal region, wherein the lead body includes a control opening at the proximal end of the curled shaft, wherein the push-pull element extends through the control opening into a control lumen within the lead body to the lead manifold at the proximal end of the lead body, wherein the lead manifold includes a tension-compression member for securing the push-pull element and providing tension and compression to the push-pull element.

6. The pacing lead of claim 5 wherein said tension-compression member generates a compressive force in said push-pull element that is able to generate an outward lead applied force of the curled shaft against the endocardial surface to enhance contact of said electrode sites.

7. The pacing lead of claim 5 wherein said curled shaft is formed with a polymer having a bending modulus that generates an outward applied force of less than 0.1 Newtons and the push-pull element applies a compressive force onto the curled shaft to generate a curled shaft applied force of about 0.6 Newtons to optimize threshold current required to form capture between the electrode site and the endocardial surface.

8. The pacing lead of claim 7 wherein the push-pull element is configured to push the distal end of the curled shaft outwards into contact with the an opposing wall of the heart chamber adjacent the proximal end of the curled shaft to provide contact of the electrode site with the endocardial surface with an optimized threshold current for said electrode site.

9. The pacing lead of claim 1 wherein the pacing lead is a unipolar lead and the electrode sites are cathode sites and the pacing lead has an anode electrode located on the endocardial surface.

10. The pacing lead of claim 9 wherein the switch box measures threshold current required to attain capture by the heart chamber of the generator signals delivered to the cathode sites, wherein the switch box is able to automatically select an optimal cathode site of the plurality of said cathode sites.

11. The pacing lead of claim 1, wherein the pacing lead is a bipolar lead wherein the plurality of electrode sites comprise a plurality of alternating cathode sites and anode sites on the pacing lead body, wherein the switch box is adapted to receive generator signals from a pulse generator and direct the generator signals to the plurality of alternating cathode sides and anode sites.

12. The pacing lead of claim 11 wherein the switch box measures threshold current required to attain capture by the animal chamber of the generator signals delivered to a pair of neighboring electrode sites of the plurality of electrode sites, wherein the pair of neighboring electrode sites form a cathode site and an anode site, and wherein the switch box is adapted to automatically select an optimal pair of the neighboring electrode sites of the plurality of electrode sites.

13. The pacing lead of claim 1 wherein the curled shaft forms a loop angle ranging from 150 to 240 degrees and comprises an outward memory force, thereby being configured to contact the endocardial surface of the heart chamber.

14. The pacing lead of claim 13 wherein the curled shaft has a curled shaft radius of curvature between about 0.05 and 3.0 cm.

15. The pacing lead of claim 1 wherein the curled shaft comprises an open loop such that the lead body distal end does not overlap with the curled shaft proximal end.

16. The pacing lead of claim 1 wherein the curled shaft comprises a closed loop such that the distal end of the pacing lead overlaps with the proximal end of the curled shaft.

17. The pacing lead of claim 1 further comprising an introducer sheath to assist in the placement of the pacing lead within the heart chamber, wherein the introducer sheath comprises an inner surface and an outer surface, wherein the pacing lead is adapted to extend distally through the introducer sheath, wherein the introducer sheath has a Touhy-Borst component attached thereto, the Touhy-Borst component providing frictional and compressive attachment of the introducer sheath relative to the pacing lead to prevent movement of the pacing lead within the chamber of the heart.

18. The pacing lead of claim 1 wherein the distal end of the lead body comprises at least one orifice, wherein the orifice is in direct fluid communication with an internal lumen of the lead body.

19. The pacing lead of claim 1 wherein the lead body has an open distal end.

20. The pacing lead of claim 5 wherein the lead body distal end has a conical tip, wherein the conical tip is attached to the push-pull element, wherein a portion of the conical tip is able to extend into the control opening thereby creating a closed loop that is configured to obviate potential for snagging cordae tendineae located in the heart chamber.

21. The pacing lead of claim 20 wherein said lead body has a recess located at a proximal end of the curled shaft near the control opening, the recess providing a location for the lead body distal end to contact during activation of the push-pull element to place the lead body distal end into contact with the control opening and providing a providing a smooth transition from the lead body to the distal end of the curled shaft.

22. A pacing lead for temporary atraumatic placement via transvascular access on an endocardial surface of a heart chamber of an animal body to deliver an electrical signal comprising:
a. a lead manifold located outside the animal body;
b. a pacing lead body connected to the lead manifold, the pacing lead body having a proximal end and a distal end, wherein the pacing lead body comprises a curled shaft at the distal end region of said pacing lead body, wherein the curled shaft has a distal end and a proximal end and a curved shaped memory for temporary placement of the curled shaft against the endocardial surface, wherein the curled shaft further comprises a plurality of electrode sites, which electrode sites are connected via electrical continuity such that at least one of the plurality of electrode sites is adapted to temporarily connect to the endocardial surface, wherein each of the plurality of electrode sites is connected to an individual conduction wire, wherein each of the plurality of conduction wires extends along the pacing lead body to connect to an individual electrode connector on the lead manifold, wherein each of the plurality of electrode connectors is individually connected via the electrode conduction wire to a plurality of electrode receptacles; and
c. a push-pull element connected to the lead body distal end, wherein the push-pull element traverses external to the lead body distal region, wherein the lead body includes a control opening at the proximal of the curled shaft, wherein the push-pull element extends through the control opening into a control lumen within the lead body to the lead manifold at the proximal end of the lead body, wherein the lead manifold includes a tension-compression member for securing the push-pull element and providing tension and compression to the push-pull element.

23. The pacing lead of claim 22 having an internal lumen extending from the proximal end to the distal end of the lead body for receiving a placement stylet.

24. The pacing lead of claim 23 wherein the placement stylet is curved.

25. The pacing lead of claim 22 wherein the plurality of electrode receptacles is contained in a switch box, wherein the switch box is adapted to receive generator signals from a pulse generator and direct the electrode generator signal to specific electrode sites.
